(19) United States
(12) Patent Application Publication
(10) Pub. No.: US 2022/0380709 A1
(43) Pub. Date: Dec. 1, 2022

(54) APPARATUS FOR FORMING COMPARTMENTS AND METHODS THEREOF

(71) Applicants: National University of Singapore, Singapore (SG); Massachusetts Institute of Technology, Cambridge, MA (US)

(72) Inventors: Arif Zainuddin Nelson, Singapore (SG); Saif Abdul Kadir Khan, Singapore (SG); Patrick Seamus Doyle, Cambridge, MA (US)

(21) Appl. No.: 17/766,034

(22) PCT Filed: Oct. 2, 2020
(86) PCT No.: PCT/SG2020/050556

§ 371 Date: Apr. 1, 2022

(30) Foreign Application Priority Data
Oct. 3, 2019 (SG) 10201909264V

(51) Int. Cl. C12M 3/06; B01J 13/00; B01L 3/00

(52) U.S. Cl. CPC C12M 23/16; B01J 13/0052; B01L 3/502; B01L 2200/027

(57) ABSTRACT

The present invention relates to a method and an apparatus for forming one or more compartments in a yield-stress fluid, wherein the one or more compartments can be one or more droplets. The yield-stress fluid is selected from polydimethylsiloxane, silicone oil, colloidal particles in water or oil, diblock or triblock copolymers in water or oil, microcellulose, xanthum gum, 0.1 wt % Carbopol and a combination thereof. The present invention is applicable for use in crystallisation, bioassays and chemical microreactors.

### TECHNICAL FIELD

The present invention relates, in general terms, to an apparatus for forming one or more compartments. The present invention also relates to methods for forming one or more compartments. The present invention is applicable for use in crystallisation, bioassays and chemical microreactors, for example.

### BACKGROUND

Traditionally, droplets are produced in large stirred batches of immiscible fluids. In their simplest form, droplets can be formed in an emulsion of two immiscible liquids (for example, oil and water) in which one liquid (the dispersed phase) is in the form of macroscopic or microscopic droplets dispersed in the other (continuous) phase. Emulsions can also contain emulsifiers to lower the interfacial tension and hence reduce the energy required to break the dispersed phase into droplets, and also preventing them from coalescing by generating a repulsive force or a physical barrier between them. However, droplets formed using this method have sizes which are usually spread over a Gaussian distribution. This is undesirable if precise control over the chemistry within the droplets is required.

Fluidic manipulation of micro- and milliscale droplets has become core to many fields and applications, including particle synthesis, chemistry, and biology. Microfluidic techniques improve on these batch methods and offer precise control over droplet composition and size by mixing fluids during flow within small diameter fixtures and tubing, or custom-fabricated devices. However, as these droplets are required to travel through fixtures and tubings or devices, the droplets are subjected to surface forces and fluid dynamics which may influence the chemistry occurring within the droplets. Further, persistent challenges and limitations remain such as the complexity of device manufacturing, a lack of flexibility with discrete channels and fixed geometries, and difficulty in selectively processing and extracting individual droplets. Efforts to address some of these challenges include the development of dynamic flow patterning, patterned substrates with “fluid walls”, and open microfluidic capillary systems, yet inherent limitations are still present due to the primary reliance of these and other microfluidic methods on phenomena and fluids that are purely Newtonian.

It would be desirable to overcome or ameliorate at least one of the above-described problems, or at least to provide a useful alternative.

### SUMMARY

In accordance with the present disclosure, there is provided a method for forming one or more compartments in a yield-stress fluid, including: a) introducing one or more volumes from an outlet of a nozzle into a yield-stress fluid, the outlet of the nozzle being in contact with the yield-stress fluid; and b) displacing the nozzle and/or the yield-stress fluid relative to each other to thereby form one or more compartments in the yield-stress fluid, the one or more compartments being formed by, or being formable from, the one or more volumes.

Advantageously, the volumes may be selectively introduced at pre-determined locations within the immiscible or partially miscible yield-stress fluid. Such volumes are embedded within the yield-stress fluid, such that the volumes are spatially isolated from each other and not influenced by solid boundaries (e.g. solid containing walls) that may influence induced internal processes.

In certain embodiments, the method further includes a step of selectively perturbing a flow of an input fluid for forming one or more volumes.

In certain embodiments, the selective perturbation of the flow of the input fluid may be synchronised to the displacement of the nozzle and/or the yield-stress fluid relative to each other.

In certain embodiments, the selective perturbation of the flow of the input fluid may be a periodic variation of the flow of the input fluid.

In certain embodiments, the one or more compartments are one or more droplets, and the one or more droplets may be suspended in the yield-stress fluid.

The method may further include a microfluidic system in communication with an inlet of the nozzle for supplying the input fluid.

The method may further include a flow system in communication with the yield-stress fluid for supplying the yield-stress fluid as a continuous flow to the outlet of the nozzle.

In certain embodiments, the one or more compartments is, or are, non-miscible with the yield-stress fluid.

In certain embodiments, the yield-stress fluid is at rest.

In certain embodiments, the yield-stress fluid is contained in a vessel.

In certain embodiments, the vessel is displaceable relative to the nozzle.

In certain embodiments, the displacement is in Cartesian coordinates.

In certain embodiments, the yield-stress fluid has a yield-stress value of about 0.1 Pa to about 10 Pa.

In certain embodiments, the yield-stress fluid has a surface tension of about 5 mN/m to about 75 mN/m.

In certain embodiments, the yield-stress fluid has a critical shear rate e,dot γ_{c} of about 0.01 1/s to 1000 1/s, and a flow index n of about 0.25 to about 1.

In certain embodiments, the yield-stress fluid has a characteristic thixotropic timescale of about 0 seconds to about 10 seconds.

In certain embodiments, the yield-stress fluid is selected from polydimethylsiloxane, silicone oil, colloidal or granular particles in water or oil, diblock or triblock copolymers in water or oil, microfibrillar cellulose, xanthan gum and a combination thereof.

In some embodiments, the yield-stress fluid is semi-transparent or transparent to allow for direct observation of the compartments.

In certain embodiments, the one or more compartments is, or are, suspended within the yield-stress fluid at least 1 mm below a surface of the yield-stress fluid.

Advantageously, this ensures that the compartments are always enclosed within the yield-stress fluid and perturbations will not cause the compartments to be displaced and escape the yield-stress fluid.

In certain embodiments, the input fluid is a liquid.

In certain embodiments, the one or more volumes in the one or more compartments is, or are, diffusible out from the one or more compartments for use in crystallisation.

In certain embodiments, each of the one or more compartments has a different composition for following progress of a chemical reaction.

In certain embodiments, each of the one or more compartments includes a microorganism for use in bioassays.

There is also provided an apparatus for forming one or more compartments, including: a) a nozzle including an outlet, the outlet for introducing one or more volumes; b) a yield-stress fluid, the yield-stress fluid in contact with the outlet of the nozzle; and c) a controller configured to displace the nozzle and/or the yield-stress fluid relative to each other to introduce one or more volumes into the yield-stress fluid to thereby form one or more compartments from the one or more volumes.

In certain embodiments, the apparatus further includes a controller configured to selectively perturb a flow of an input fluid for forming one or more volumes.

In certain embodiments, the selective perturbation of the flow of the input fluid is synchronised to the displacement of the nozzle and/or the yield-stress fluid relative to each other.

In certain embodiments, the selective perturbation of the flow of the input fluid is a periodic variation of the flow of the input fluid.

The apparatus may further include a microfluidic system in communication with an inlet of the nozzle.

In certain embodiments, the yield-stress fluid is contained in a vessel.

### BRIEF DESCRIPTION OF THE DRAWINGS

Embodiments of the present invention will now be described, by way of non-limiting example, with reference to the drawings in which:

FIG. 1 is a flow diagram of a method for forming one or more compartments;

FIG. 2 schematically illustrates how the compartments are formed in a direct writing method;

FIG. 3 illustrates an example of compartments (mineral oil) formed in a yield-stress fluid (Carbopol in water) via a direct-writing setup;

FIG. 4 illustrates a prior art example of compartments of ethyl acetate forming in a solution of polyvinyl alcohol in water (not a yield-stress fluid); both the ethyl acetate compartments and PVA solution are deposited into a xanthan gum yield stress fluid;

FIG. 5 shows a side-view image sequence of droplet pinch-off with a nozzle, and various parameters that can be regulated to obtain compartments in a method according to certain embodiments;

FIG. 6 illustrates a top-down view of printed droplets following an example of a process described herein;

FIG. 7 presents (a, b) examples of crystals forming within compartments in a yield-stress fluid; and (c) an example of recovering the crystals by collapsing the yield-stress fluid;

FIG. 8 illustrates another embodiment of the present invention when used in the crystallisation of compounds;

FIG. 9 illustrates scanning electron micrograph images of crystals forming using a direct-writing method and deposition method according to certain embodiments;

FIG. 10 illustrates (a) an array of compartments formed by mixing multiple components at the point of injection, with each compartment having different chemical composition; (b) a line of aqueous compartments (blue, top) embedded in an oil-based yield stress fluid and subsequently injected with an various amounts of a second input fluid (red, bottom);

FIG. 11 illustrates various ways in which materials can be added and/or removed from the compartments;

FIG. 12 illustrates a pictorial representation of a method for forming an array of compartments, the compartments containing nanoparticle precursors and the resultant nanoparticle containing compartments;

FIG. 13 illustrates an example and results of a bioassay containing bacterial cells, with citric acid mixed within the input fluid; and

FIG. 14 illustrates an example and results of a bioassay containing bacterial cells, with citric acid subsequently added to the compartments;

FIG. 15 shows another embodiment of the present invention when used in a bioassay;

FIG. 16 provides a summary of the techniques that can be used with the present invention; and

FIG. 17 is a schematic of an apparatus for forming compartments according to certain embodiments;

FIG. 18 illustrates continuous embedded droplet printing setup on a benchtop and a schematic representation of the print;

FIG. 19 plots incipient shear stress of powder flow as a function of the applied normal stress;

FIG. 20 plots the rheology of 0.1% Carbopol 980 model yield-stress fluid;

FIG. 21 shows spherical crystallized particles produced by embedded droplet printing and through microfluidic crystallization with Newtonian fluids; and

FIG. 22 shows images of another embodiment of the present invention.

### DETAILED DESCRIPTION

As used herein, ‘yield-stress fluid’ is a material that reversibly transitions from effectively solid to effectively fluid and back again as a consequence of applied mechanical stress. It is a material that is solid-like below a critical stress but undergoes a dramatic drop in viscosity and flows above its yield stress. Yield-stress fluids are generally considered non-equilibrium systems both in their solid-like state and their tendency to age and flow over long timescales. Yielding may be gradual across a range of stress magnitudes and may occur as localized brittle cracking, shear-banding, or system spanning diffuse failure. An important distinction is that of the ‘static’ yield stress (for the transition from solid to liquid, e.g. start-up shear tests) versus the ‘dynamic’ yield stress (for the transition from liquid to solid, e.g. steady flow tests of decreasing shear rate). These can be very different; when they are, the static yield stress is typically larger. Furthermore, the forward and reverse transitions between solid-like and liquid-like are not instantaneous; the microstructural units require time to return to an arrested state. This reversible time-dependence is known as thixotropy and it is intimately linked with yielding in these materials. For practical purposes, ‘simple yield-stress fluids’ may be defined as those for which thixotropic timescales are too short to observe with available techniques while ‘thixotropic yield-stress fluids’ are those with a measurable time scale.

At least some embodiments of the invention have advantages over batch techniques. These advantages include precise control of small sample volumes and high frequency of automated generation. Furthermore, the bath material (yield-stress fluid) provides an intrinsic barrier against atmospheric contamination or evaporation of the fluid samples. At least some embodiments of the invention can also overcome some of the disadvantages of traditional microfluidic technologies by removing the aspects of solid boundaries (e.g. tubing and fixtures) and continuous flow. Microfluidic device designs exist for trapping many droplets in a large reservoir or in arrays of chambers but droplet mobility may make indexing difficult and the presence of shared boundaries may lead to fouling or affect processes like nucleation. By embedding compartments within a yield-stress fluid, compartments can be processed in spatial isolation for essentially an indefinite period of time with no risk of coalescence or collapse, barring some degradation of the bath. This is not possible with traditional microfluidics since droplets are generally constrained by flow through the length of a given microfluidic device. For example, in some microfluidic cases, the flow may be paused but there is a significant risk of continued droplet mobility and droplet coalescence. It has been suggested that droplets in microfluidic devices may be circulated in very long tubing to develop. However, this was found to be challenging to execute because droplets will become unstable and collapse or coalesce due to the non-negligible hydraulic resistance.

To this end, embodiments of the present invention are suitable for the generation, trapping, and processing of fluid droplets within yield-stress fluids. By printing discrete droplets that are embedded within a bath of yield-stress fluid, the droplets are not limited to fixed geometric boundaries, and can freely manipulate and process selected droplets for extended periods of time, and there is no complex device manufacturing. Furthermore, droplets embedded in yield-stress fluids are not affected by any solid boundaries or convective effects which are often inherent to other forms of microfluidics. This results in droplets with no mobility or risk of coalescence whatsoever, even without utilizing surfactants.

Rayleigh instability is the phenomenon where a falling stream of fluid breaks up into smaller packets with the same volume but less surface area. The driving force of Rayleigh instability is that liquids, by virtue of their surface tensions, tend to minimize their surface area. In the past, a considerable amount of work has been done to reduce or avoid this fluid instability in, for example, 3D printing in sacrificial yield-stress fluid baths as it introduces inconsistencies in a technique which strives for precision and control. Advantageously, the inventors have found that by embracing Rayleigh instability, which would otherwise be problematic in many contexts, droplets of controllable size and volume can be formed in a yield-stress fluid. The use of a yield-stress fluid to support and maintain the droplets further removes aspects of solid boundaries and continuous flow, thus providing a reproducible environment substantially removed from external forces and influences.

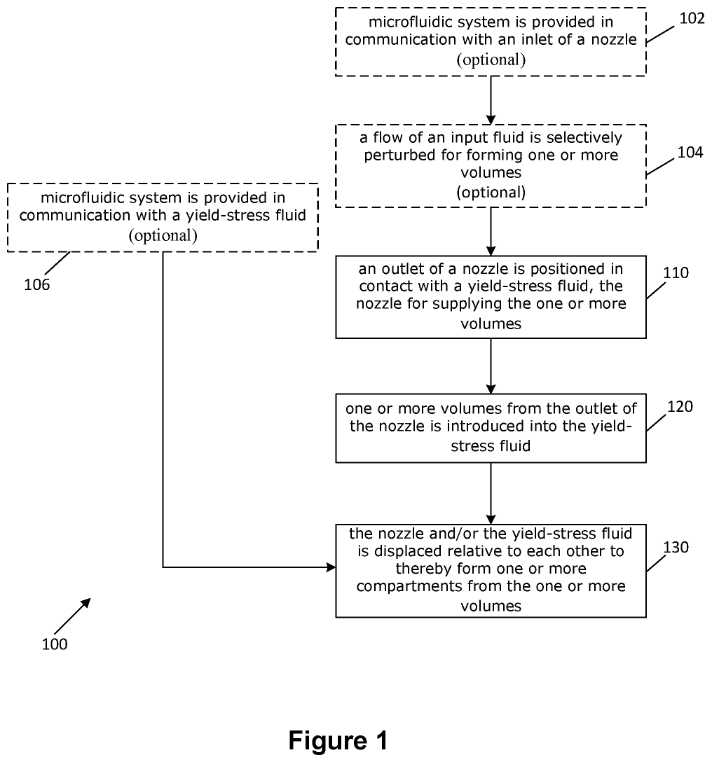

FIG. 1 is a flow diagram of an example process 100 of forming one or more compartments.

At step 110, an outlet of a nozzle is positioned in contact with a yield-stress fluid. The nozzle is suitable for supplying one or more volumes of input fluid to the yield-stress fluid. At step 120, the one or more volumes exit from the outlet of the nozzle and are introduced into the yield-stress fluid. At step 130, the nozzle and yield-stress fluid are displaced relative to each other. In this regard, the nozzle can be moved while the yield-stress fluid is stationary, or the yield-stress fluid can be moved while the nozzle is stationary, or both the nozzle and yield-stress fluid can be moved. The displacement allows for the formation of one or more compartments in the yield-stress fluid, the one or more compartments being formed from the one or more volumes.

The nozzle is optionally in communication with a fluid reservoir, and with a pump that can be used to introduce one or more volumes of fluid from the nozzle into the yield-stress fluid. For example, optional step 102 can include providing a microfluidic system in communication with an inlet of the nozzle. The microfluidic system can be modulated to provide the one or more volumes. Alternatively, optional step 104 can include selectively perturbing a flow of the input fluid for forming one or more volumes.

The yield-stress fluid is optionally in communication with a fluid reservoir. The fluid reservoir is for supplying a flow of yield-stress fluid to the nozzle. In this regard, the yield-stress fluid is moved/translated while the nozzle is stationary. For example, optional step 106 can be a microfluidic system provided in communication with the yield-stress fluid for displacing the yield-stress fluid relative to the nozzle. Preferably, the yield-stress fluid is provided as constant flow to the nozzle.

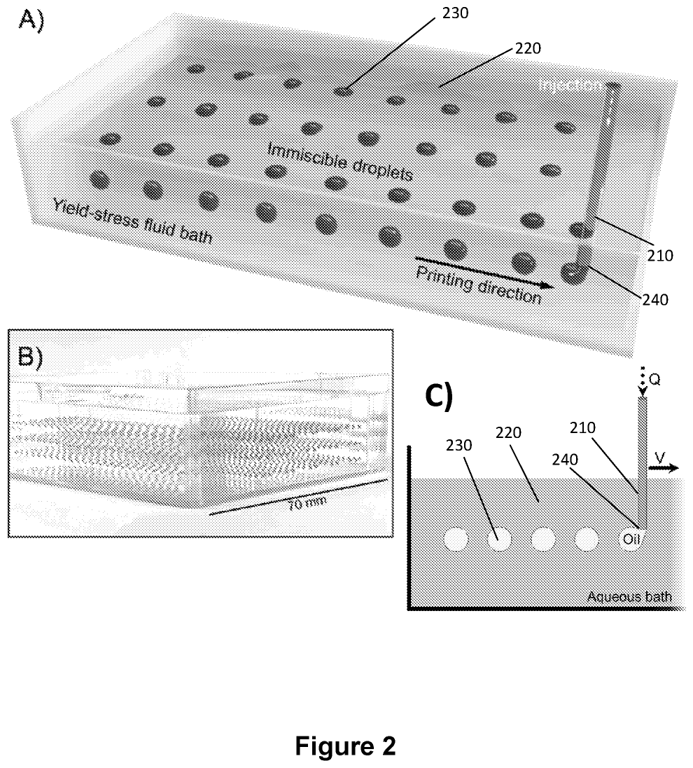

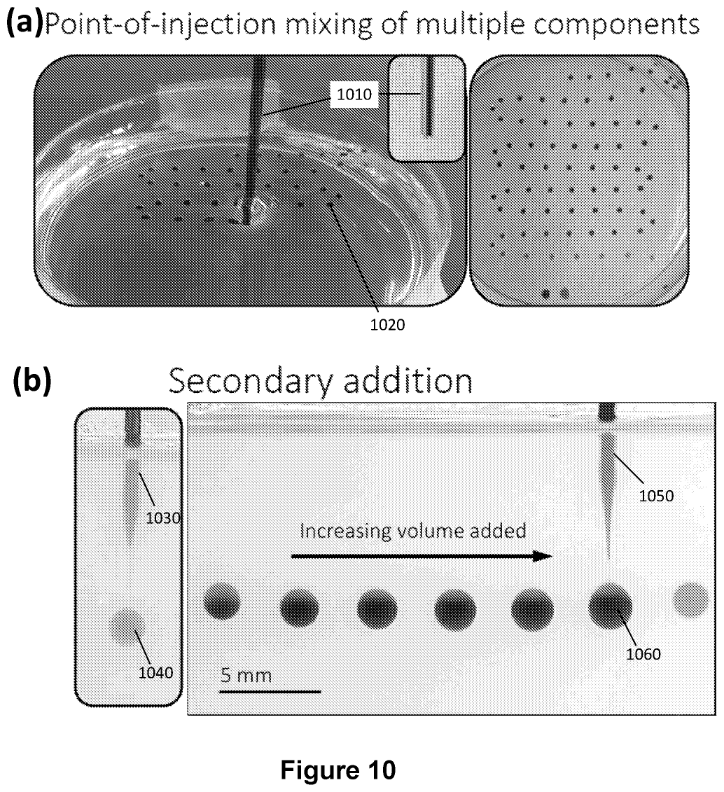

FIG. 2(a) illustrates a schematic of a method according to an embodiment of the present invention. A submerged nozzle 210 may be translated through a bath of yield-stress fluid 220 while injecting an immiscible fluid to produce suspended compartments 230. The size, shape and arrangement of the compartments can be controlled in a 3-dimensional space. As shown in FIG. 2(b), a 30-by-10-by-3 array of compartments (containing oil) that are 1 mm in diameter is suspended in a water-based yield-stress fluid. The bath possesses rheological properties that suspend the compartments in place with no influence from exterior convective forces or solid surfaces. The yield-stress fluid bath provides a barrier to contamination and allows for precise and complex experimental procedures to be performed. The bath yields due to the motion of the nozzle, and surface tension between the now-fluidized bath and injected fluid causes breakup of the injected fluid into spherical droplets. A syringe pump can for example be coupled to a motorized stage to print arrays of highly uniform droplets with a predictable volume in precise locations as shown in FIG. 2(b). These droplets are embedded within the bath with no risk of coalescence or collapse without the use of surfactants and remain at their programmed locations for an essentially indefinite period of time— barring some triggered transformation of the bath material. This allows for performing complex experimental processing of the droplets in a selective manner as exemplified by the addition of precise volumes of a secondary fluid shown in FIG. 10(b). FIG. 2(c) illustrates a side-view schematic of how the compartments are formed in this direct writing method. A nozzle (fluid injection device) 210 is shown and the outlet 240 of the nozzle is in contact with the yield-stress fluid (aqueous bath). As the nozzle 210 is displaced in the direction and rate as indicated by V, one or more volumes from the outlet 240 of the nozzle is introduced into the yield-stress fluid 220 to form the one or more compartments 230. In this case, the compartments contains an oil and Q is the volumetric flow rate of the input fluid. To provide the one or more volumes, a flow of an input fluid can be selectively perturbed.

As used herein, ‘compartment’ refers to an enclosed pocket of a substance. This substance can be a liquid and/or a gas. When the compartment is, or contains, a liquid or both a liquid and a gas, the compartment may be referred to as a droplet. When the compartment only contains a gas, the compartment may be referred to as a bubble. As will be shown herein, the compartment can contain chemical compounds, biological materials such as cells, genetic materials and viruses, crystals, nanoparticles precursors and/or nanoparticles.

The perturbation of the input fluid to allow for the formation of the volumes and compartments can be by stop-flow setup, which can be manually operated or programmed to release a volume of input fluid periodically. Alternatively, the volumes and compartments can be formed by relying on Rayleigh instability.

The rheology of the yield-stress fluid bath determines the theoretical operating space for the present invention. The compartments are generated by yielding the yield-stress fluid to an inserted nozzle that injects fluid. As the nozzle and yield-stress fluid are displaced relative to each other, the localised displacement applies a localised mechanical stress to the yield-stress fluid, thus converting the localised volume to transit into a fluid. When the volume exits the nozzle, the fluid state yield-stress fluid allows for the formation of a compartment. As the yield-stress fluid reverts back to the solid state, the compartment is held in place, embedded at a pre-determined location within the yield-stress fluid. Compartments are formed when the surface tension between the injected fluid and the yield-stress bath obeys the relation (γ/r) σ_y (surface tension, γ, divided by the radius of the compartment, r, is greater than or equal to the bath yield stress, σ_y).

Without wanting to be bound by theory, comparing the yield stress of the bath, σγ, to the stress exerted on the bath by a spherical droplet of diameter, d, due to buoyancy from a density difference, Δρ, the equation obtained is

$$\frac{1}{2} π {d}^{2} {σ}_{Y} > \frac{1}{6} π {d}^{3} Δ ρ g {Y}_{crit} \qquad ( 1 )$$

where g is the gravitational acceleration constant, and Y_{crit} is a dimensionless parameter defined as the ratio of the fluid's yield stress to the stress exerted on the fluid by the droplet when motion occurs due to a yielding transition. The definition of Y_{crit} was rearranged to describe the operating space of interest. One might naively expect Y_{crit} to be unity for yielding to occur, but due to the droplet needing only to yield a finite volume to move, theoretical studies have found a value of Y_{crit}=0.14 for an idealized fluid; experiments have shown Y_{crit} to be material specific, ranging between 0.05 and 0.6. Equation 1 is the condition for a formed droplet to be statically suspended within the yield-stress fluid bath rather than sedimenting or floating due to buoyancy. Comparing the yield stress to the stress exerted due to surface tension γ, for a curved interface with diameter d, we obtain

$${σ}_{Y} < \frac{2 γ}{d} . \qquad ( 2 )$$

This inequality is the condition for whether droplet formation will occur. If the yield stress of the bath is too great, a continuous thread of the injected phase will form rather than discrete droplets. With these conditions, an operating space for embedded droplet printing for a pairing of a model yield-stress fluid and an immiscible phase can be determined.

In some embodiments, the yield-stress fluid has a Y_{crit} of about 0.05 to about 1, or preferably about 0.05 to about 0.6. In other embodiments, Y_{crit} is about 0.1 to about 0.6, about 0.1 to about 0.5, about 0.1 to about 0.4, about 0.1 to about 0.3, or about 0.1 to about 0.2.

The present invention allows for the manipulation of droplets under conditions that are simply unattainable with conventional microfluidic methods, namely the elimination of exterior influences including convection and solid boundaries. Advantageously, the compartments are held for an effectively indefinite period of time in an “absolutely quiescent” state with a complete absence of exterior convective forces (including buoyancy-driven flows), solid boundaries that may influence induced internal processes (e.g. solid containing walls) and continuous flow. Further, there is no possibility for cross-contamination between compartments since they are always spatially isolated, and there are no shared solid boundaries that can lead to fouling or uncontrolled nucleation. The present invention also removes some troublesome aspects of microfluidics including the use of surfactants and the complexity of device manufacturing.

Additionally, as Rayleigh instability generates the volumes as a periodic function and amplitude, the compartments formed are consistent in size and shape. The compartments are also maintainable without the use of emulsifiers and surfactants.

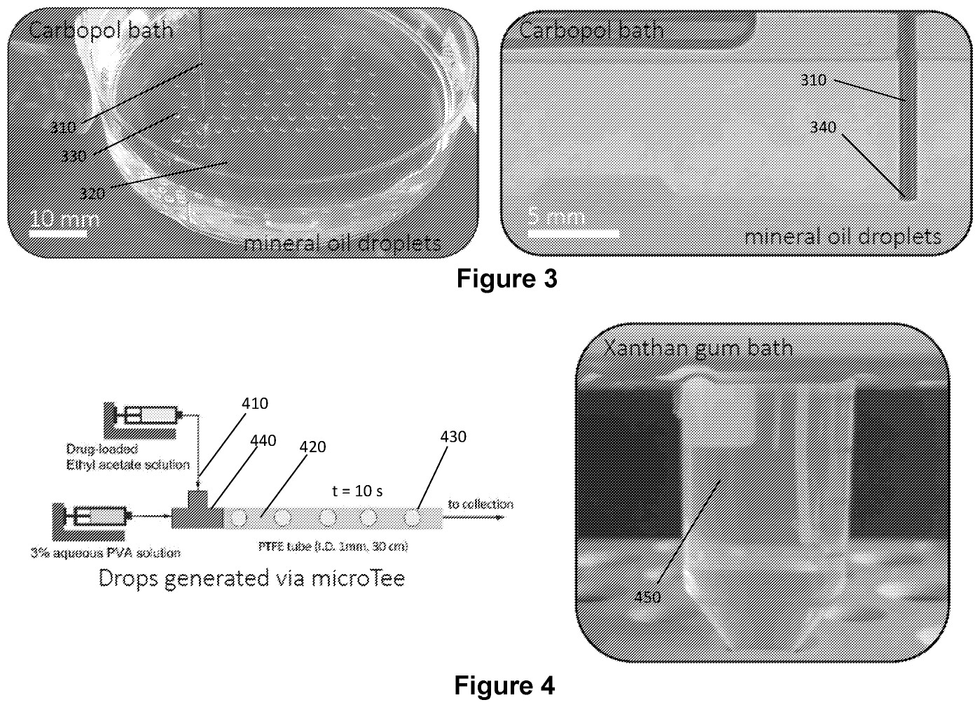

FIG. 3 shows compartments 330 being formed in an embodiment of the present invention. In this example, the compartments 330 (mineral oil) are formed in a direct-writing setup; i.e. the nozzle 310 is displaced relative to the yield-stress fluid 320 (Carbopol in water) by moving the nozzle 310. As the nozzle 310 transits to the next location, the volume from the nozzle breaks off, forming a compartment 330 at the former location. In this way, by continuously moving the nozzle in a stationary yield-stress fluid, multiple compartments can be formed and deposited within the yield-stress fluid 320.

In contrast, FIG. 4 depicts an example used in the prior art. In this example, a continuous phase 420 containing surfactant (polyvinylalcohol; PVA) is displaced relative to the nozzle 410 by moving the continuous phase. The output of the nozzle is in contact with the continuous phase 420 within the microTee component 440. As the continuous phase flows past the nozzle, the flow of input fluid out from the nozzle forms one or more volumes, which forms the compartments 430 (ethyl acetate) on entering the continuous phase 420. The compartments are flowed into a collection medium 450 (bath), which can be a yield-stress fluid (for example xanthan gum). In this way, the pre-form compartments are deposited into the yield-stress fluid bath.

However, the disadvantage with this method is that the continuous phase is constantly being added to the yield-stress fluid. This can cause the yield-stress fluid to gradually lose its thixotropic property (for example). The inability to maintain a consistent environment in the yield-stress fluid can result in a gradual change in compartment morphology and/or content. In some scenarios, the compartments can collapse on entering the yield-stress fluid. Further, the dilution of the yield-stress fluid by the continuous phase can cause the yield-stress fluid to lose its solid-like state such that the compartments cannot be selective positioned. In contrast, the present invention allows for the maintenance yield-stress fluid such that the compartments are homogenous throughout the deposition method. The present invention also allows for selective placement of the compartments.

The selective perturbation of the flow of the input fluid may be synchronised to the displacement of the nozzle and the yield-stress fluid relative to each other. This synchronisation enables compartments of a consistent size and shape to be formed in the yield-stress fluid. Further, the synchronisation also reduces and/or eliminates the formation of satellite compartments; i.e. smaller sized compartments formed at the tail end of the primary compartment. Additionally, by timing the relative movement of the nozzle and the yield-stress fluid, due to surface tension, any formed satellite compartments are able to coalesce with the primary compartment, thus eliminating the presence of the satellite compartments which may be undesirable. Such synchronisation would be dependent on several factors, for example the properties of the yield-stress fluid and the rheology of the input fluid.

In certain embodiments, the selective perturbation of the flow of the input fluid may be a periodic variation of the flow of the input fluid.

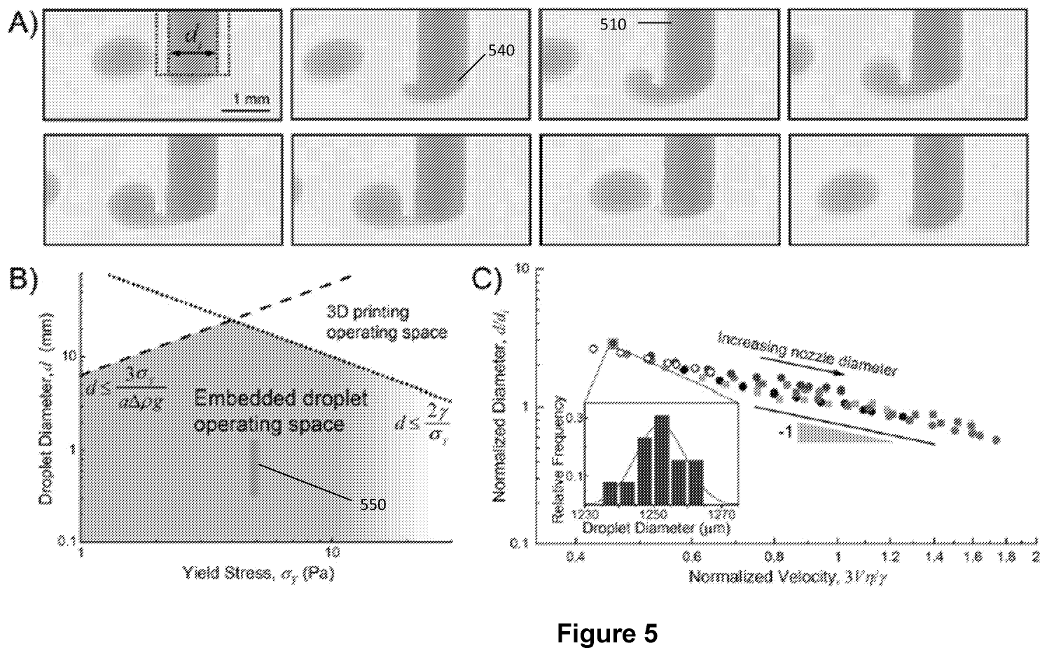

FIG. 5(A) shows a side-view image sequence of droplet pinch-off from the outlet 540 with the nozzle 510 translating towards the right-side of the frame. A mineral oil is being extruded into an aqueous yield-stress fluid (0.1 wt % Carbopol) at a volumetric flowrate of 50 μl/min from a hydrophilically treated glass tube moving at a speed of 200 mm/min. Dotted lines are overlaid on the boundaries of the glass tube for clarity. The depicted sequence occurs over a period of approximately 800 milliseconds. Carbopol is an aqueous jammed suspension of poly(acrylic acid) microgels. Carbopol is generally considered a “simple” yield-stress fluid (short thixotropic restructuring time) and is also optically transparent. FIG. 5(B) illustrates that the theoretical operating space for embedded compartment printing is determined by capillary forces and gravitational settling. This is distinct from the operating space used for embedded 3D printing. The equations shown are calculated for the case of mineral oil in a water-based yield-stress fluid. Assuming the surface tension for this system does not deviate significantly from that of water with mineral oil and using a value of 0.3 for Y_{crit}for Carbopol, Eqs. 1 and 2 result in the operating space depicted in FIG. 5(B). Other theoretical boundaries can also be drawn. In particular, and for example, a high-concentration Carbopol suspensions (higher yield stress and postyield viscosities) can also be used with further optimisation (for example, by decreasing wetting of the nozzle by the injected fluid) to reduce inconsistent droplet generation. FIG. 5(C) illustrates some embodiments that fall within the highlighted region 550 of FIG. 5(B). FIG. 5(C) shows that nondimensionalization yields a master curve in the operating range of interest. Nondimensionalized droplet diameter and velocity for a set of mineral oil droplets are printed in Carbopol from cylindrical nozzles made of steel (circle symbols) of nominal inner diameter 0.21 mm (open circles), 0.41 mm (gray circles), 0.44 mm (dark gray circles), 0.72 mm (black circle), and 1.5 mm (light gray circles); or hydrophilically treated glass (square symbols) of nominal inner diameter 0.67 mm (light gray squares), and 1 mm (dark gray squares). All droplets were produced using a volumetric flowrate of 50 μl/min. The inset histogram depicts the relative frequency of absolute droplet diameter for the indicated experimental point. The solid line is a Gaussian fit with σ/μ=0.006.

As shown in FIG. 5(A), the dispersed phase grows at the nozzle exit and tends to fill the void left behind as the nozzle translates relative to the bath (toward the right). A neck forms at the edge of the nozzle outlet which further thins, and the droplet detaches from the nozzle. This process is similar to droplet breakoff in coflowing streams of Newtonian fluids, wherein droplets detach when the streamwise forces exceed the force due to interfacial tension.

In certain embodiments, the one or more compartments are one or more droplets. The one or more compartments may be suspended in the yield-stress fluid. Advantageously, and in addition to the removal of external forces, suspending the compartments allows them to be visualised in a 3D environment. In this regard, changes in the internal chemistry within the compartment can be tracked and sampled at any time point.

The velocity of the nozzle relative to the bath was systematically varied for different nozzle diameters and a fixed injection flow rate of 50 μL/min. The nozzle materials were also varied between stainless steel and glass that has been plasma cleaned shortly before droplet generation to be hydrophilic and thus more readily wet by the bath phase. The droplet size obtained as a function of velocity for droplets that range in diameter from about 300 μm to about 1.5 mm. The generated droplets are highly uniform in size, as depicted in FIG. 5(C). The insert shows a representative data point, the coefficient of variation, σ/μ, is 0.6%. The frequency of droplet pinch-off translates to a linear density of embedded droplets and a resulting spatial resolution. Across all experiments depicted in FIG. 5(C), the maximum linear density observed is 0.83 droplets per millimeter. The spacing between rows and layers of printed droplets can also be controlled such that a maximum volumetric density of 66.4 droplets per milliliter of yield-stress fluid is achieved.

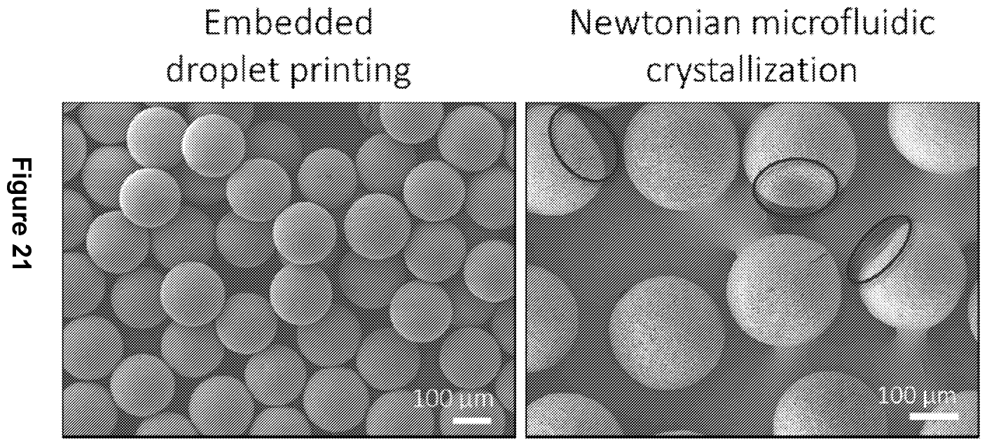

The analysis of the conventional, purely Newtonian case of droplet formation from coflowing streams balances the viscous Stokes' drag with the interfacial tension. This results in a normalized droplet size, d/di, where di is the inner diameter of the nozzle; and a capillary number, 3Vη/y, where V is the velocity of the nozzle relative to the bath; η is the non-Newtonian viscosity of the yield-stress fluid bath at a shear rate of V/dₒ, where dₒ is the outer diameter of the nozzle (see FIG. 20 for bath rheology). Using these non-dimensional variables that include the non-Newtonian viscosity, we obtain a master curve for this material system that has a power-law scaling of −1 as depicted in FIG. 5(C). A subset of the nozzles was verified that increasing the injection flow rate shifts the master curve slightly upward, but the overall scaling is preserved. Thus, the variables derived for the Newtonian case can be simply modified to allow for varying the size of resulting droplets in the system. It is expected that the present invention will also work for a wider range of different flow rates, nozzle geometries (e.g., tapered), or bath rheology and microstructure.

In some embodiments, a diameter of the compartment is related to the displacement rate of the nozzle and/or the yield-stress fluid relative to each other by a power-law scaling of about −1.

The compartments can have a diameter of about 50 μm to about 1.5 mm, or about 100 μm to about 1.5 mm, about 150 μm to about 1.5 mm, about 200 μm to about 1.5 mm, about 250 μm to about 1.5 mm, about 300 μm to about 1.5 mm, about 400 μm to about 1.5 mm, about 500 μm to about 1.5 mm, about 600 μm to about 1.5 mm, about 700 μm to about 1.5 mm, about 800 μm to about 1.5 mm, about 900 μm to about 1.5 mm, or about 1 mm to about 1.5 mm.

The compartments can be printed with a linear density of about 0.1 droplets per millimeter to about 1 droplets per millimeter. The compartments can be printed with a volumetric density of about 1 droplets per milliliter of yield-stress fluid to about 70 droplets per milliliter of yield-stress fluid.

The method may further include providing a microfluidic system in communication with an inlet of the nozzle for supplying the input fluid. The method may also include providing/placing a microfluidic system in communication with the yield-stress fluid. As shown in FIGS. 1 to 3, the microfluidic system can be in communication with the nozzle, for providing the input fluid, and/or in communication with the yield-stress fluid. Alternatively, the nozzle and/or the yield-stress fluid may be connected to one or more fluid delivery devices such as an extruder or syringe pump. The fluid delivery devices control the flowrates of the injected fluid. By varying the modulating system, compartments of selectable sizes can be produced. For example, by varying the flowrates of the pumping device, the volume to be injected can be varied and accordingly the compartment size can be increased or decreased.

The method may further include providing a system in communication with the yield-stress fluid for supplying the yield-stress fluid as a continuous stream to the outlet of the nozzle. The system can be a microfluidic system. In this regard, the yield-stress fluid is displaced with respect to the nozzle and the nozzle is stationary. As shown in

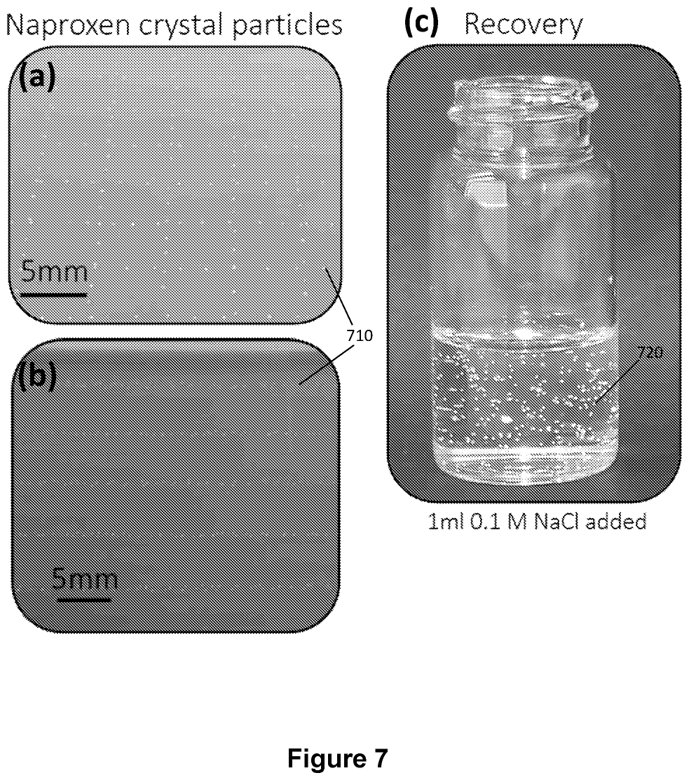

FIG. 7(b), a deposition method (similar to FIG. 4 but replacing the continuous phase 420 with a yield-stress fluid) with a yield-stress fluid in contact and in fluid communication with the outlet of the nozzle allows for the compartments to be carried away after its formation. In this setup, the yield-stress fluid (as it is flowed from an initial stock/source) can, for example, be held in a vessel positioned downstream relative to a stationary nozzle. As the yield-stress fluid is used at the outset and in the flow system, there is no change in the physical properties of the yield-stress fluid when it finally enters a bath or vessel. This also allows for selective placement of the compartments in the bath.

For the compartment to be suspended in the yield-stress fluid, some degree of immiscibility is advantageous. Immiscibility is the property where two substances are not capable of combining to form a homogeneous mixture; i.e. the two substances (oil and water) will separate from each other. In certain embodiments, the one or more compartments is non-miscible with the yield-stress fluid. As discussed above, by accounting for the surface tension of the compartments and the density difference, the compartments can be maintained as an enclosed reaction environment which is suitable for use as a chemical microreactor. When the compartments are arranged in, for example an array, the reaction condition or reaction progress can be tracked over time.

In other embodiments, the one or more compartments is, or are, partially miscible with the yield-stress fluid. In this regard, partially miscible substances can be substances that do not mix in all proportions at all temperatures; i.e. they are miscible only in a limited range of concentrations. A partially miscible substance (or compartment) can be made up of one or more components, one component of which can be miscible and the other component immiscible (hydrophobic compound in ethyl acetate). In this way, when the two substances are combined (compartment in yield-stress fluid), the miscible component of a first substance (ethyl acetate) is able to diffuse and mix with a second substance (Carbopol in water), leaving the immiscible component (hydrophobic compound) behind. This allows for the use of the compartments as a crystallisation platform, for example via an anti-solvent process.

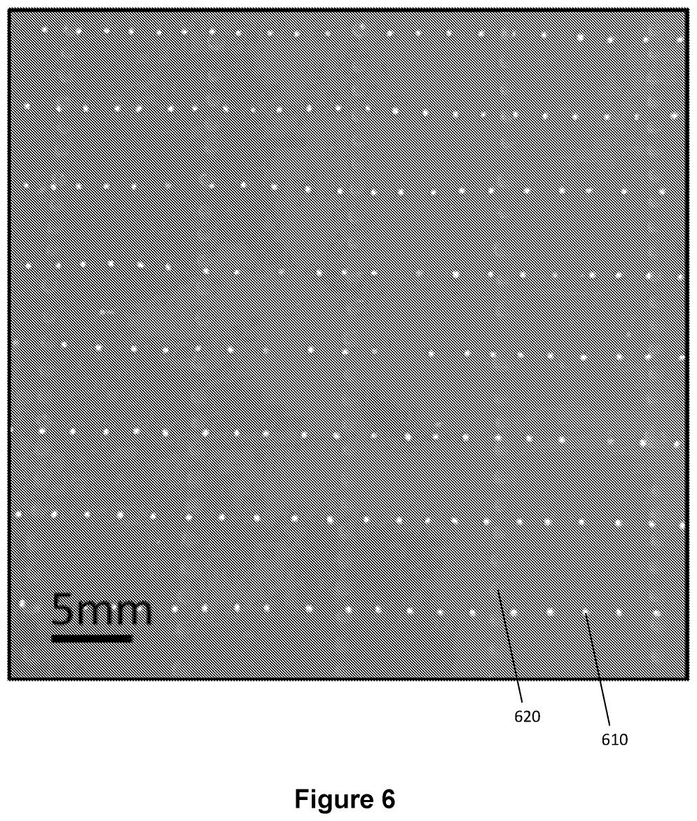

FIG. 6 illustrates a top-down view of printed compartments (610 and 620) following use of a method according to certain embodiments. The compartments contain a hydrophobic compound, which is caused to crystallize over time due to extraction of ethyl acetate into the water-based yield-stress fluid. Compartments 610 in a horizontal line pattern were printed first and have already crystallized into spherical crystal particles; subsequent droplets 620 were printed in vertical line patterns several millimeters above the horizontal line patterns and have not yet crystallized. FIGS. 7(a) and 7(b) further illustrate formed compartments 710 which are partially miscible with the yield-stress fluid. In this example, another hydrophobic compound is dissolved in ethyl acetate and used as the input fluid to form the compartments. The yield-stress fluid is Carbopol in water. By allowing the compartments to incubate in the yield-stress fluid over time, ethyl acetate is able to diffuse out from the compartments, thus forming a concentrated solution of hydrophobic compound in the compartment, favouring crystallisation. This can be visualised by the appearance of white dots, which signifies the solidification of the compound.

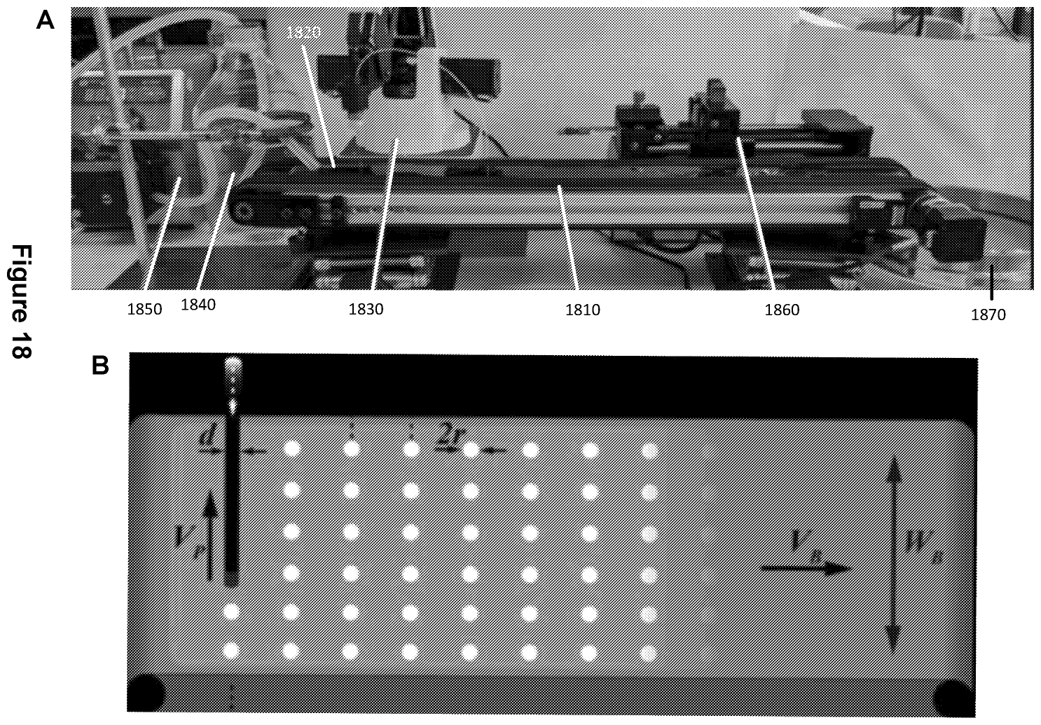

In certain embodiments, the yield-stress fluid is at rest. In certain embodiments, the yield-stress fluid is contained in a vessel. In certain embodiments, the nozzle is displaceable relative to the vessel. Alternatively, the vessel is displaceable relative to the nozzle. For example, as shown in FIG. 18, both the nozzle and the vessel (conveyor belt surface) are simultaneously displacing in different directions. The belt carries the thin film of fluid toward the right, while the nozzle displaces into and out of the page.

In certain embodiments, the displacement is in Cartesian coordinates. In some embodiments, the yield-stress fluid is mounted on an X, Y-axis motion-controlled stage and one or more nozzles mounted on a Z-axis motion controller. The motion controllers can be programmed or operated to move in a determined pattern at a pre-determined and controlled movement rate.

The yield-stress fluid may be water-based or oil-based depending on the input fluid and/or compartment formed. For example, the yield-stress fluid can be water-based if the input fluid is oil-based and vice versa. It is further advantageous if the yield-stress fluid has low opacity (and/or good clarity) for being able to visualise changes in the compartments in applications such as microreactors. To improve the consistency of the yield-stress fluid and to minimize false readings, the yield-stress fluid can be subjected to a vacuum prior to introducing the one or more volumes for removing gases/air bubbles.

In certain embodiments, the yield-stress fluid is selected from polydimethylsiloxane, silicone oil, colloidal particles in water or oil, diblock or triblock copolymers in water or oil, microfibrillar cellulose, xanthan gum and a combination thereof. For example, water-based yield-stress fluids can be made up of appropriate amounts of xanthan gum, micro-fibrillated cellulose and/or microgels of polyacrylic acid. Examples of oil-based yield-stress fluid systems include mixtures of polydimethylsiloxane, silicone oil, and hydrophilically modified fumed silica nanoparticles; and mixtures of diblock and triblock copolymers in mineral oil.

Additives can also be added to the yield-stress fluid to alter some of the properties of the yield-stress fluid. In some embodiments, the rheological additives do not interact with the compartments. In this regard, the additives do not affect with the interior processes within the compartments. In some embodiments, additives are added at less than 5% wt/wt, or less than 4% wt/wt, or less than 3% wt/wt, or less than 2% wt/wt, or less than 1% wt/wt.

In some embodiments, the yield-stress fluid is composed of additives that are generally regarded as safe by the United States Food and Drug Administration. In some embodiments, the yield-stress fluid is selected from Carbopol in water, fumed silica in silicone oil and polydimethylsiloxane (PDMS). Carbopol is a polyacrylic acid microgel sold by Lubrizol in powdered form. This would be considered a system of swollen granular particles (˜10 μm diameter) in water. This system is transparent and can be triggered to lose its yield stress through the shrinkage of the granular particles. Fumed silica can be for example Aerosil 200 (pharmaceutical grade sold by Evonik). Silicone oil can for example be obtained from Sigma Aldrich and PDMS purchased from Dow Chemical Company. Fumed silica in silicone oil and polydimethylsiloxane would be considered a system of colloidal particles in oil. This system is semi-transparent. Further advantageously, when PDMS is caused to be crosslinked by addition of a crosslinking agent, this system can be triggered to permanently gel. This allows the compartments to be entrapped within for ease of transportation. In some embodiments, the yield-stress fluid is about 0.07 wt % to about 1 wt % Carbopol.

For example, the yield-stress fluid can be selected from:

a) 0.1 wt % Carbopol (polyacrylic acid microgels) in water;
b) 4 wt % fumed silica, 48 % silicone oil. 48 % PDMS;
c) 0.1 wt % xanthan gum in water;
d) 0.4 wt % microfibrillar cellulose in water;
e) 2.25 wt % diblock, triblock copolymer, 95.5 % mineral oil;
f) 1-2wt % Laponite in water;
g) 15-20 wt % Pluronic F127 in water; and
h) 60-70 wt % silicone oil in water emulsion.

Other examples of yield-stress fluids are shown in the table below (but not limited to):

| Material Class | Material System Details/Additive | Yield Stress Range | |
|---|---|---|---|
| Crowded hard particles | Brownian: PMMA spheres, silica | 1-500 | Pa |
| | sphere mixtures, anisotropic polystyrene | | |
| | Non-Brownian: polystyrene | 1-1000 | Pa |
| | beads, glass beads, sand | | |
| Effectively-crowded | Laponite in water | 10-500 | Pa |
| charged particles | | | |
| Crowded soft particles | Carbopol microgels in water, poly-NIPAM | 0.1-200 | Pa |
| | microgels in water, polybutadiene stars, | | |
| | dendrimers, diblock and triblock copolymer | | |
| | mixture in oil | | |
| Crowded micelles | Pluronic F127 in water, poly(oxyethylene- | 0.1-500 | Pa |
| and vesicles | oxybutylene) in water, Triton X-100 in water | | |
| Crowded emulsions | Silicone oil-in-water stabilized with SDS, | 0.03-1000 | Pa |
| | water-in-mineral oil stabilized with Span-Tween | | |
| Foams | Nitrogen in microgel solution, nitrogen in | 0.1-20 | Pa |
| | water/glycerol stabilized with TTAB, dodecanoyl | | |
| Particulate gels | Bentonite in water, silica in organic solvents, | 0.06-500 | Pa |
| | titanium dioxide in water, carbon black in | | |
| | oil, metal oxide nanoparticles in water, | | |
| | rod-shaped virus particles in water | | |
| Electrorheological fluids | Titanium dioxide in silicone oil, silica | 0 (no applied | |
| | spheres in corn oil | field) −120 kPa | |
| Magnetorheological | Carbonyl iron in mineral oil, | 0 (no applied | |
| fluids | carbonyl iron in grease | field) −80 kPa | |
| Associating | Nanofibrillated cellulose in water, | 1-500 | Pa |

| | |
|---|---|
| polymers or fibrils | Xanthan gum in water |

To control the compartments size and placement in the yield-stress fluid, the following properties of yield-stress fluid and other engineering properties can be modulated (but not limited to):

a) yield stress (Pa);

b) elastic modulus pre-yield, G (Pa);

c) yield strain (%);

d) thixotropic restructuring time (s);

e) post-yield viscosity (Pa s) or viscous effects e.g. Herschel-Bulkley parameters:

critical shear rate, flow index;

f) uniaxial strain at break (%);

g) density (kg/m³);

h) homogeneity of the material (particle, building block, or aggregate size) (pm);

i) surface energy and wetting;

j) optical properties, e.g. maintain light transmittance for photo-chemical responsiveness; and

k) transformation requirements, e.g. chemical or thermal.

Some of the engineering properties of yield-stress fluids considered in the present invention include the yield-stress value, the post-yield viscous effects [i.e., Herschel-Bulkley parameters], optical properties, surface energy and wetting, and the ability to trigger mechanical changes in the yield-stress fluid.

In some embodiments, the yield-stress fluid can be characterised by the Herschel-Bulkley model. A Herschel-Bulkley fluid is a generalized model of a non-Newtonian fluid, in which the strain experienced by the fluid is related to the stress in a complicated, non-linear way. Three parameters characterize this relationship: the critical shear rate, \dot{γ}_{c}, the flow index n, and the yield shear stress Tₒ. The critical shear rate is the rate of deformation at which the flow stress is twice the yield stress, while the flow index measures the degree to which the fluid is shear-thinning or shear-thickening. Finally, the yield stress quantifies the amount of stress that the fluid may experience before it yields and begins to flow. Alternatively, a proportionality constant, k, may be used in place of \dot{γ}_{c}, where k =τ₀/(\dot{γ}_{c})ⁿ.

In some embodiments, the critical shear rate \dot{γ}_{c} is about 0.01 1/s to 1000 1/s. In other embodiments, the critical shear rate \dot{γ}_{c} is about 0.1 1/s to 1000 1/s, about 1 1/s to 1000 1/s, about 10 1/s to 1000 1/s, about 50 1/s to 1000 1/s, about 100 1/s to 1000 1/s, or about 500 1/s to 1000 1/s.

In some embodiments, the flow index n is about 0.25 to about 1. In other embodiments, the flow index n is about 0.3 to about 1, about 0.4 to about 1, about 0.5 to about 1, about 0.6 to about 1, about 0.7 to about 1, or about 0.8 to about 1.

In some embodiments, the yield shear stress To is about 0.1 Pa to 100 Pa. In other embodiments, the yield shear stress To is about 0.1 Pa to 100 Pa, about 1 Pa to 100 Pa, about 10 Pa to 100 Pa, about 50 Pa to 100 Pa, or about 80 Pa to 100 Pa.

The yield stress of the yield-stress fluid is selected to be large enough to prevent the compartments from settling to the bottom or floating to the top. If the yield stress is too high or if the surface tension is too low, a continuous thread of fluid will form rather than discrete compartments. If the yield stress is too high or if the nozzle does not have sufficient affinity (wettability) for the bath, the injected fluid may wet the nozzle and flow back upwards. If the yield stress is too high or the printing depth is too shallow, a static crevice will be created by the motion of the nozzle through the bath. Such are not desirable.

In certain embodiments, the yield-stress fluid has a yield-stress value of about 0.1 Pa to about 10 Pa. In other embodiments, the yield-stress fluid is about 0.2 Pa to about 10 Pa, about 0.3 Pa to about 10 Pa, about 0.5 Pa to about 10 Pa, about 1 Pa to about 10Pa, about 2 Pa to about 10 Pa, about 3 Pa to about 10 Pa, about 4 Pa to about 10 Pa, about 5 Pa to about 10 Pa, about 6 Pa to about 10 Pa, about 1 Pa to about 9 Pa, about 1 Pa to about 8 Pa, about 1 Pa to about 7 Pa, about 1 Pa to about 6 Pa, about 2 Pa to about 6 Pa, or about 2 Pa to about 5 Pa.

In certain embodiments, the yield-stress fluid has a surface tension of about 5 mN/m to about 75 mN/m. Some examples of fluids with surface tensions within this range are water in ethyl acetate (6.8 mN/m), water in mineral oil (50 mN/m) and air in water (73 mN/m).

In certain embodiments, the yield-stress fluid has density of about 800 kg/m³ to about 1000 kg/m³. Alternatively, the density can be about 800 kg/m³ to about 950 kg/m³, 800 kg/m³ to about 900 kg/m³ or 800 kg/m³ to about 850 kg/m³.

In certain embodiments, the yield-stress fluid has a characteristic thixotropic timescale of about 0 seconds to about 10 seconds. Alternatively, the characteristic thixotropic timescale can be about 1 s to about 10 s, about 2 s to about 10 s, about 3 s to about 10 s, about 3 s to about 9 s, about 3 s to about 8 s or about 3 s to about 7 s.

In some embodiments, the yield-stress fluid is semi-transparent or transparent to allow for direct observation of the compartments. In this regard, the yield-stress fluid allows light to pass through so that objects within can be partially or distinctly seen.

The one or more compartments is, or are, suspended at a distance below the surface of the yield-stress fluid. In certain embodiments, the one or more compartments is, or are, suspended within the yield-stress fluid at least 1 mm below a surface of the yield-stress fluid. In other embodiments, the distance is at least a diameter of the one or more compartments. In other embodiments, the distance is at least twice the diameter of the one or more compartments. In this regard, the compartments are printed sufficiently far from the boundaries of the container or from the surface of the fluid to avoid contact with the air-liquid interface or solid-liquid interface. Advantageously, this ensures that the compartments are totally embedded within the yield-stress fluid and are less likely to be perturbed by external forces. This also provides a buffer distance and ensures that the compartment is maintained within the yield-stress fluid when subjected to sudden and unintentional movement.

As the output of the nozzle is at the interface of the volume and the yield-stress fluid, the chemical and physical properties of the nozzle can also influence the formation of the compartments. For example, factors such as the geometry, surface energy and wettability of the nozzle, movement rate of the nozzle, and injected flowrate of the input fluid can affect the compartment size.

For modifying the wettability of the nozzle, the nozzles can be coated with polydimethyl siloxane (hydrophobic), coated with poly vinyl alcohol (hydrophilic), and/or plasma cleaned (hydrophilic).

In some embodiments, the water surface energy is about 5 mN/m to about 100 mN/m. In other embodiments, the surface energy is about 5 mN/m to about 800 mN/m, or about 5 mN/m to about 50 mN/m.

In some embodiments, the nozzle has an output diameter of less than 2 mm, or less than 3 mm, less than 4 mm, less than 5 mm, less than 10 mm or less than 20 mm. In other embodiments, the nozzle has a movement rate of about 50 mm/min to about 4000 mm/min, about 50 mm/min to about 3800 mm/min, about 50 mm/min to about 3500 mm/min, about 50 mm/min to about 3200 mm/min, about 50 mm/min to about 3000 mm/min, about 60 mm/min to about 3000 mm/min, about 70 mm/min to about 3000 mm/min, about 100 mm/min to about 3000 mm/min, about 200 mm/min to about 3000 mm/min, about 500 mm/min to about 3000 mm/min, about 750 mm/min to about 3000 mm/min, about 1000 mm/min to about 3000 mm/min or about 1500 mm/min to about 3000 mm/min. In other embodiments, the flowrate of the input fluid is about 1 μL/min to about 3000 μL/min, about 2 μL/min to about 3000 μL/min, about 5 μL/min to about 3000 μpL/min, about 10 μL/min to about 3000 μL/min, about 20 μL/min to about 3000 μL/min, about 50 μL/min to about 3000 μL/min, about 70 μL/min to about 3000 μL/min, about 100 μL/min to about 3000 μL/min, about 150 μL/min to about 3000 μL/min, about 200 μL/min to about 3000 μL/min, about 500 μL/min to about 3000 μL/min, about 750 μL/min to about 3000 μL/min, about 1000 μL/min to about 3000 μL/min, about 1000 μL/min to about 2500 μL/min, or about 1500 μL/min to about 2000 μL/min.

In certain embodiments, the input fluid is a liquid. In other embodiments, the input fluid is a gas.

The compartments formed using this presently disclosed method can function as “microreactors” for many different processes and applications related to crystallization, biology, or chemical reactions.

The present invention can be used in crystallisation of spherical drug particles. In the manufacturing of pharmaceuticals, common secondary manufacturing processes are drug crystallization, followed by milling, and granulation. The goal of these processes is to obtain a flowable drug powder for further processing into a final tablet. The present invention can be used to combine the three aforementioned processes into one step through spherical crystallization with a high degree of control over key attributes including size, shape, and structure by use of evaporative means or the careful choice of partially miscible solvents (anti-solvent crystallization). In certain embodiments, the input fluid in the one or more compartments is diffusible out from the one or more compartments for use in crystallisation.

FIGS. 6 and 7 shows how the present invention can be used in a crystallisation process. For the yield-stress fluid, a mixture consisting of polyacrylamide microgels such as Carbopol 980 from Lubrizol in water at a concentration of between 0.05 and 0.25 wt % can be used. Other water-based formulations have also been tested and are found to be also suitable. For the input fluid, hydrophobic drug compounds such as Naproxen or Fenofibrate can be dissolved in ethyl acetate at a concentration of between 50-500 mg/ml. After compartment (610, 620 and 710) formation within the water-based yield-stress fluid, ethyl acetate is able to diffuse into the yield-stress fluid to leave a super saturated solution of the drug compound in the compartment which favours crystallization. In this regard, by allowing the compartments to incubate for a certain amount of time, crystals of drug compounds can be formed (for example in compartment 610). FIG. 6 depicts a two-layer grid of printed compartments during a time point of the anti-solvent crystallization process, via the extraction of ethyl acetate into the water-based yield-stress fluid. The horizontally aligned compartments 610 have crystallized and accordingly are observed to be opaque spheres. The uniform size and spacing of the particles is notable and is due to the constant movement rate of the nozzle relative to the yield-stress fluid. Compared to the horizontal compartments, the vertically aligned compartments 620 were printed as a layer several millimetres above the layer of horizontal compartments and have not had sufficient time to crystallize. FIG. 7(a) illustrates crystals formed by the direct-write method, i.e. the injected input fluid is the compound in ethyl acetate. FIG. 7(b) illustrates crystals formed by the deposition method, i.e. the injected phase is preformed compartments containing the compound, the compartments preformed in yield-stress fluid (ethyl acetate in PVA solution). The injected phase is then further flowed into a bath containing yield-stress fluid. After crystallization, the crystals 720 may be recovered by adding a sufficient amount of a salt such as sodium chloride to the yield-stress fluid to increase the electrolyte concentration, thus collapsing the microgel structure and eliminating the yield stress so that the particles will settle to the bottom of the container and recovered (FIG. 7(c)). It was found that as the particles are isolated, secondary nucleation is suppressed. The crystals are also homogenous and highly spherical as there is no influence from gravitational settling or convective forces.

In the production of pharmaceutical tablets, it is often necessary to have a flowable powder of crystallized active pharmaceutical ingredient (API) and excipient. Granules of agglomerated crystals often have poor flowability in large part due to irregular shapes and wide size distributions, with many recent batch methods for crystallizing particles often still resulting in only what may be described as “approximately spherical” at best. Microfluidic approaches to spherical crystallization offer significant improvements in this regard, but crystals are often still marred by the deforming effects of viscous drag and interactions with other particles or solid surfaces, leading to noticeable deviations from sphericity (see FIG. 21 for comparisons) or unintended nucleation. Furthermore, microfluidic approaches require the use of surfactants which are often an unwanted ingredient in a final powder product.

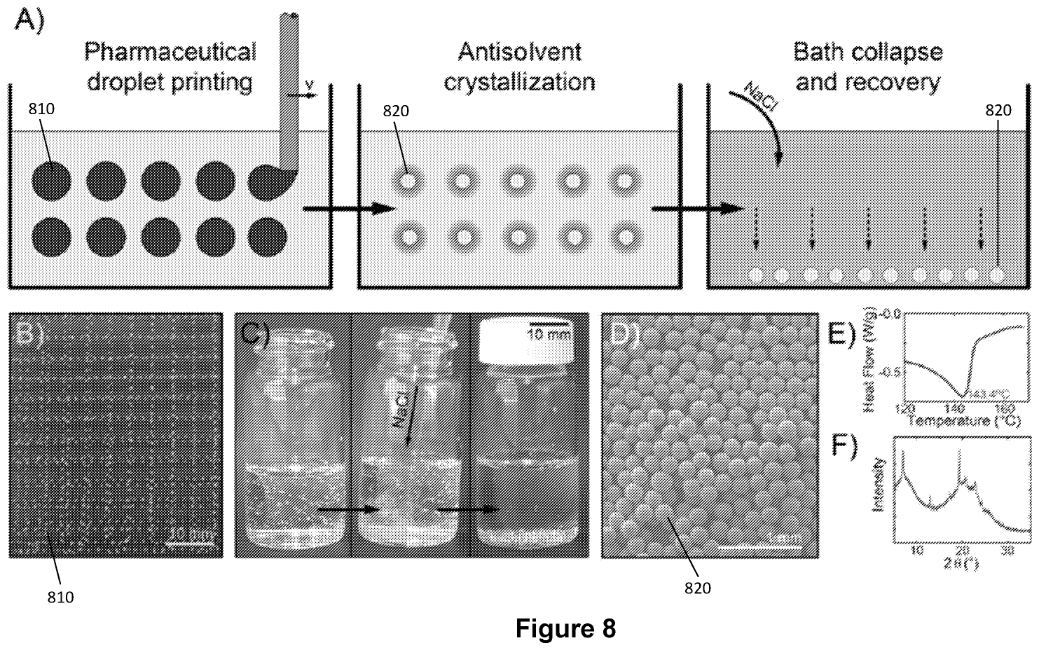

Embodiments of the present invention allows for crystallization to occur under absolutely quiescent conditions, by utilizing an antisolvent crystallization approach to produce uniform spherical particles inside aqueous Carbopol yield-stress fluid as depicted in FIG. 8(A). FIG. 8 is another example of crystallization and shows embedded droplets 810 allowing for “absolutely quiescent” crystallization of spherical particles. Droplets of ethyl acetate containing dissolved hydrophobic API and excipient can be printed. The droplets are spatially isolated with no risk of coalescence or collapse even though there are no surfactants present in either the droplet or the bath phase. The ethyl acetate is extracted into the aqueous bath since it is partially miscible in water, leaving behind a supersaturated API—excipient solution which crystallizes as shown in FIG. 8(B). For an initial droplet diameter of 800 μm, the characteristic diffusion time (square of diameter divided by diffusivity) for ethyl acetate in water is ˜10 min. In these experiments, the volume of ethyl acetate extracted into the bath is less than 1% of the total bath volume, and thus no change in the bath rheology due to dilution that would likely occur for significant or repeated injected volumes was observed. Since the initial printed droplets are highly uniform and spherical due to Equation 2 being satisfied, the crystallized particles share these characteristics. With embedded droplet printing, the extraction of ethyl acetate occurs purely diffusively, with no influence from viscous drag that may deform the droplet or from solid surfaces that would affect the nucleation process; i.e. an environment in which droplets are spatially isolated from each other and solid surfaces and in which there are no exterior convective flows. Because of this, it is believed that this approaches the ideal, completely undisturbed, conditions for terrestrial production of crystallized particles that are as spherical and uniform as possible, key targets in the manufacturing of pharmaceutical materials.

As shown in FIG. 8(A), the crystals can be collected after some time. After printing of an appropriate pharmaceutical drug solution, antisolvent crystallization results in solid spherical particles 820 that can be recovered after triggering the bath material structure to collapse. The compartments 810 are initially printed as shown in FIG. 8(B), with droplets suspended in 0.1wt % Carbopol that contain Naproxen, a model hydrophobic drug, and ethyl cellulose, an excipient. When a small volume of 1M NaCI being added to a 0.1wt % Carbopol bath with initially suspended crystallized drug particles, within 60 seconds after being agitated, the Carbopol microstructure has collapsed, eliminating the yield-stress property and allowing for recovery and washing of the particles 820 (FIG. 8(C). For this process, ˜200 mg/h of particles in 300 mL of yield-stress fluid (i.e., 0.67 mg·h⁻¹·mL⁻¹ yield-stress fluid) was produced. Field emission scanning electron microscopy image of Naproxen and ethyl cellulose drug particles produced via the sequence described above are shown in FIG. 8(D). Compared to typical spherical crystallization techniques, embedded droplet printing produces highly uniform particles with no obvious common shape defects. Further studies using differential scanning calorimetry thermogram (FIG. 8(E)) and X-ray powder diffraction plot (FIG. 8(F)) of the particles depicted in FIG. 8(D) indicate the crystallinity of Naproxen of form 1.

Accordingly, in some embodiments, the yield-stress fluid is capable of a triggered change in mechanical properties such as the loss of its yield stress properties.

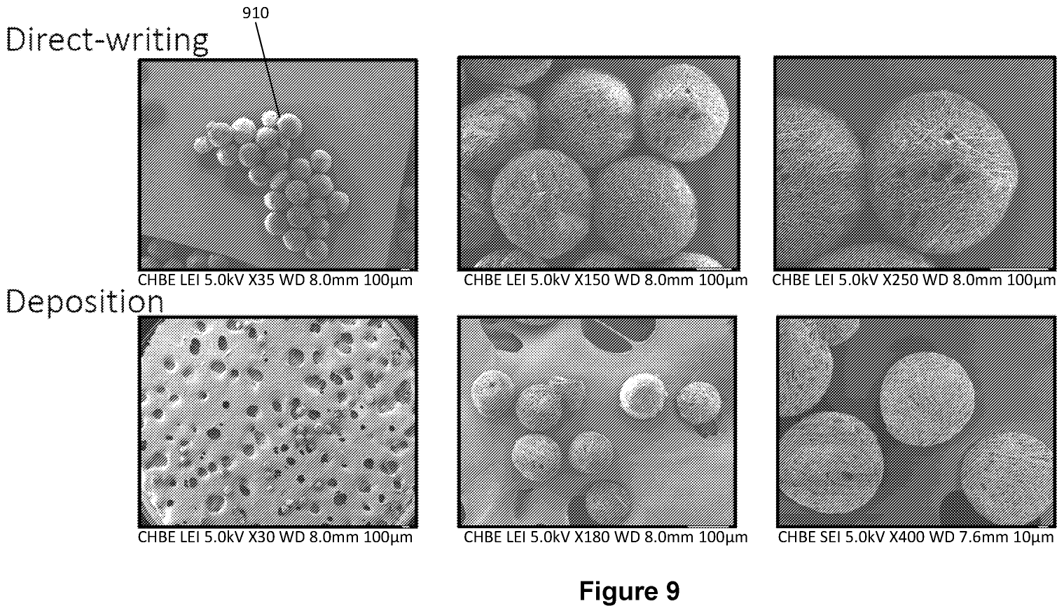

FIG. 9 shows scanning electron micrograph images of the drug compound crystals 910 formed using the two different setups as disclosed herein; i.e. direct writing and deposition. By completely eliminating exterior convective forces, the present invention allows for more perfectly spherical samples.

In certain embodiments, the one or more compartments each has a different composition for following a progress of a chemical reaction.

FIG. 10 shows examples of the present invention being used as chemical microreactors. The microreactors can be arrayed such that the progression of the chemical reaction can be tracked over various conditions. FIG. 10(a) shows a method of mixing multiple components at the point-of-injection of the nozzle 1010. In this example, the nozzle 1010 comprises two channels in parallel for providing two different fluids to form the compartment. The channels are separately in fluid communication with separate microfluidic system or pumping system, thus allowing different amounts of the fluids to be incorporated into each compartment 1020. In this way, the array can comprise compartments, each compartment having a different composition. This is illustrated in FIG. 10(a) using fluids of different colours. The first formed compartment in the array has a 100% composition of a first fluid while the last formed compartment in the array has a 100% composition of a second fluid. The compartments formed between the first and last compartments have a variable composition of the first and second fluids.

FIG. 10(b) shows another way of setting up an array with each compartments having a variable composition. Rather than mixing components before injection, an alternative setup is to have a first input fluid to form the compartments 1040 from a first nozzle 1030. A second input fluid from a second nozzle 1050 can be subsequently incorporated into the compartments 1060. The second nozzle 1050 can be the first nozzle 1030 or another nozzle. The second input fluid can have a controllable volume which is proportional to the compartments that have already been printed. Initial compartment printing can be performed with a nozzle that has an affinity for the bath material. Subsequent addition of a second input fluid can be performed with a second nozzle that has an affinity for the printed compartment material. FIG. 10(b) shows first formed dyed aqueous compartments suspended in an oil-based bath and a hydrophilically modified nozzle injecting increasing volumes of another dyed aqueous solution. In using this method, the proximity of the initial compartments to each other and the boundaries of the bath should be taken into account to avoid the compartments merging with each other. By utilizing subsequent injection, the present invention allows for complex experimental scheduling or manufacturing processes to be performed simply and on an individual compartment basis.

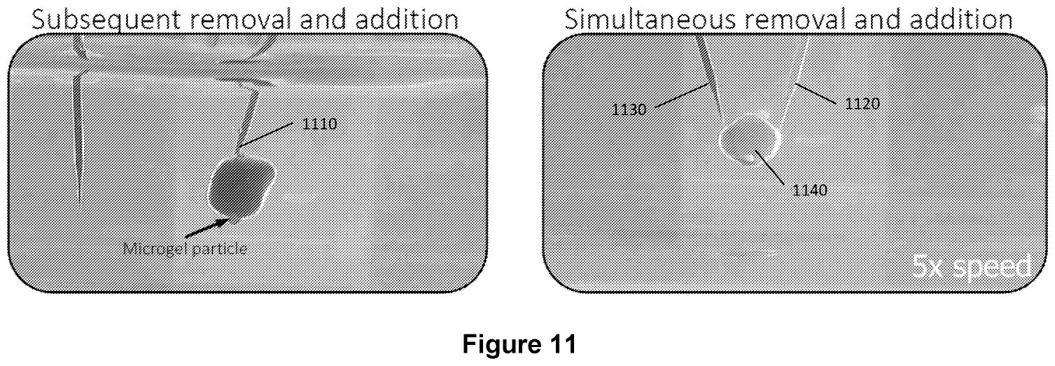

FIG. 11 shows an example performing reactions within the compartment. Fluid and/or material from the compartments can be subsequently extracted via nozzle 1110. This allows for characterization if desired. Fluids and/or materials in the compartment can also be simultaneously removed via nozzle 1120 and added via nozzle 1130 to the compartment 1140.

The present invention allows for the ability to perform chemical reactions in high throughputs and with small and precise volumes of potentially expensive reagents. By further removing interactions with solid boundaries and external flow, considerably more freedom in arraying droplets is obtained. Embedded droplet printing eliminates reported challenges in droplet microfluidics such as droplet coalescence and collapse, reduces the risk of fouling in fixed geometries, and easily allows droplets to develop for extended timescales and be selectively addressed or directly sampled.

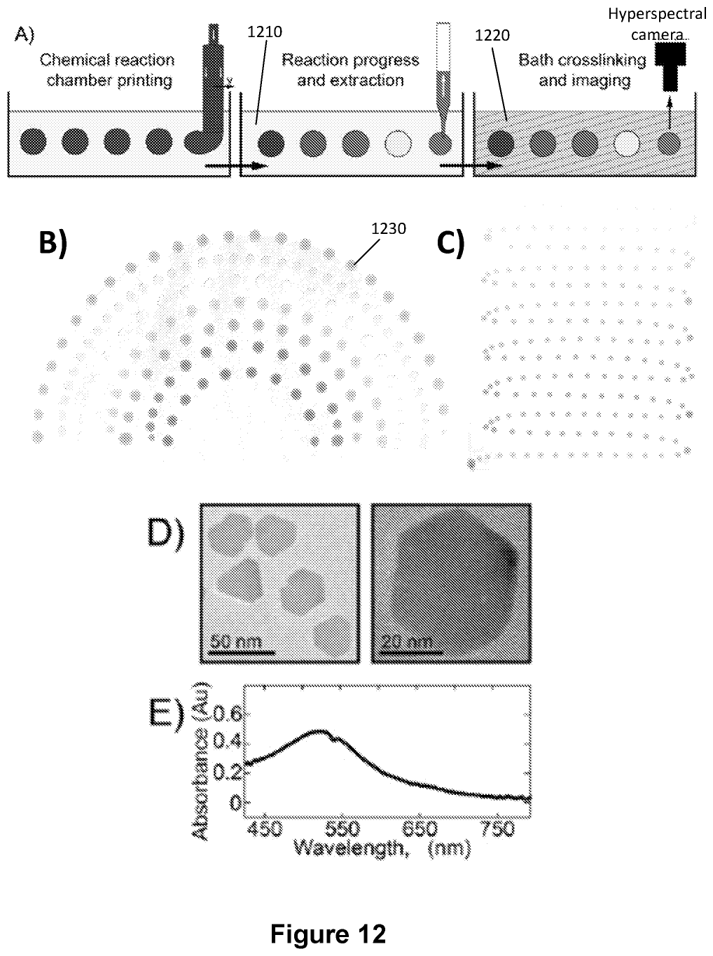

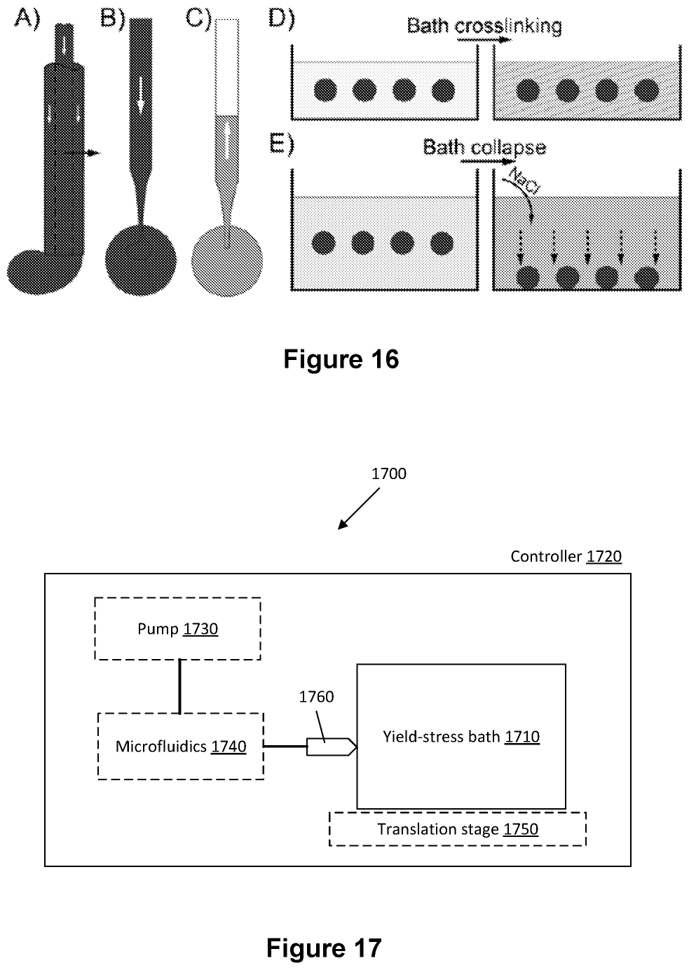

Embodiments of the present invention can provide a versatile platform for performing micro-batch chemical reactions. Precisely controlled quantities of multiple reagents may be simultaneously injected and allowed to react. After the reaction has progressed, fluid may either be directly extracted, optically characterized or both in sequence. FIG. 12 illustrates another example of compartments with varying compositions generated to allow chemical reactions to occur over long times simultaneously and with small volumes of the injected fluids. Here, seed-mediated growth of silver nanoparticles is exemplified. By mixing aqueous seed solution and AgNO₃ in different proportions, nanoparticles grow to different sizes and shapes and produce different visible optical signatures. For the injected fluids, varying ratios of aqueous silver nitrate and a “seed solution” were mixed (nanoparticles seeds, reducing agents, and stabilizers) using a microtee fixture while maintaining a constant total flowrate (FIG. 12(C)). For example, a point-of-injection mixing via concentric nozzles (FIG. 16(A)) to correlate input flow rates to the droplets being generated at a given time and position, as well as reduce the risk of fouling (compared to mixing in a T-junction fixture or similar process) can be used. Alternatively, discrete jumps in the injected formulation are also possible (FIG. 12(B)). The bath 1210 is oil-based and consists of a mixture of 48 wt % SYLGARD 184 silicone elastomer from Dow Chemical Company, 48 wt % 10 cSt silicone oil from Sigma Aldrich, and 4 wt % hydrophilically modified fumed silica nanoparticles from Evonik. Just before use, a small amount of SYLGARD 184 curing agent can be added to the bath to permanently solidify 1220 it after the compartment printing. This may be aided with low heating. Addition of the curing agent shortly before printing does not affect compartment generation but permanent solidification and trapping of the droplets allows for easier handling and imaging of the material during characterization as there is no danger of yielding the bath. The array of embedded compartments 1230 several days after printing (as depicted in FIG. 12) shows varying coloured compartments, which is evidence of differently sized nanoparticles having grown within each compartment. This also demonstrates that continuous or discrete change the proportions of reactants by controlling the flow rates of attached syringe pumps can be achieved. Further analysis can be performed (for example by using a hyperspectral camera), such as absorbance, from the embedded compartments. Embedded droplets containing silver nanoparticles produced by the described method may be characterized by transmission electron microscopy as shown in FIG. 12(D), which shows images of silver nanoparticles extracted from two different embedded droplets; or by spectral imaging as shown in FIG. 12(E) which shows the absorbance spectrum of a representative embedded droplet.

In many conventional microfluidic systems, procedures that require significant reaction times often have significant drawbacks. For example, flow and droplet generation might be paused as a reaction proceeds, limiting throughput and risking the merging of still-mobile droplets; droplets might also be circulated through very long or looping tubing, but this reportedly risks coalescence and collapse. Alternatively, droplets could be deposited into a large reservoir or a trapping device that may be complex to manufacture; these techniques typically randomize the droplet placement inside sealed devices, making indexing and extraction difficult. Using embedded droplet printing, such limitations are removed or at least minimized, thus greatly facilitating time-lapse studies.

Further advantageously, products can be directly extracted from droplets of interest for characterization such as with transmission electron microscopy (TEM) as shown in FIG. 12(D), verifying that nanoparticle shape and size vary for different droplet compositions; this kind of selective extraction remains challenging in conventional microfluidic systems. By transforming the yield-stress fluid into a permanent solid material, the handling and additional characterization of embedded droplets is also facilitated. As depicted in FIG. 16(D), gentle heating triggers the bath material to cross-link, fixing the droplets in place. No change in colour of the droplets was observed during this heating process. The bath material remains semitransparent after cross-linking and the droplets can be optically characterized in situ via a hyperspectral camera. FIG. 12(E) shows representative absorbance spectra. The spectra are peak shifted compared to a purely spherical silver nanoparticle case, indicating the presence of anisotropic nonspherical particles as expected and as seen in the TEM images. Without cross-linking of the bath material, this process would have been difficult; due to the low yield stress of the bath material, transportation of the entire bath is challenging without droplets shifting position and potentially merging.

Accordingly, in some embodiments, the yield-stress fluid is capable of a triggered change in mechanical properties such as permanently gelling.

In this regard, the method can further include a step of collapsing or gelling the yield-stress fluid for collecting or handling the compartments.

The present invention can also be used to investigate gaseous chemical reactions. In this regard, the compartments contain gases and the yield-stress fluid are impervious to the gases being tested. Advantageously, this allows for small volumes of gases to be used, which can be beneficial if the gaseous reagents are expensive, flammable and/or explosive.

Embedded droplet printing allows for small volume bio-assays with directly accessible droplets. In certain embodiments, the one or more compartments each includes a microorganism for use in bioassays.

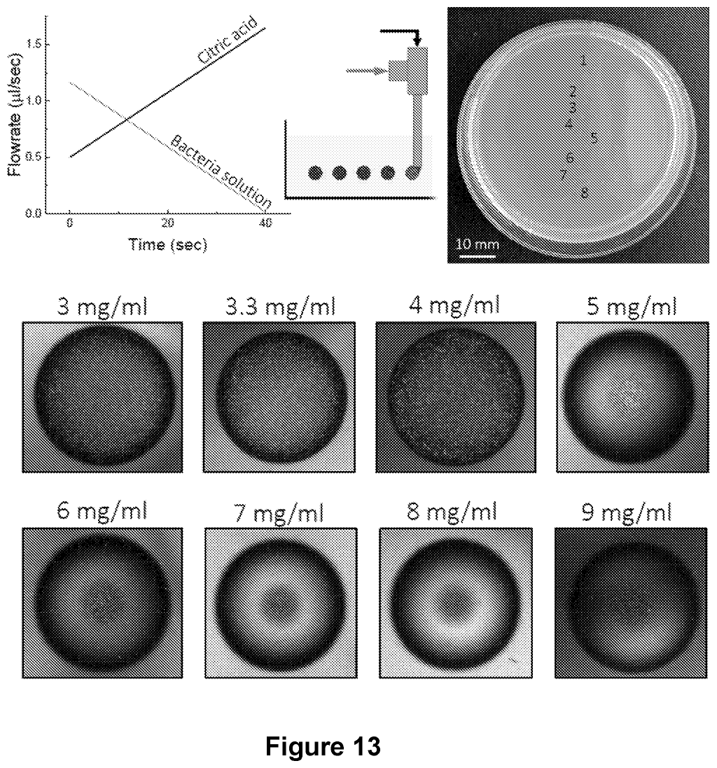

FIG. 13 illustrates an example in which the compartments are used to assay bacteria in bioassays. The injected fluid is a growth media containing bacteria (P. aeruginosa in growth media mixed with varying amounts of citric acid). The yield-stress fluid is a mixture of 48 wt % SYLGARD 184 silicone elastomer from Dow Chemical Company, 48 wt % 10 cSt silicone oil from Sigma Aldrich, and 4 wt % hydrophilically modified fumed silica nanoparticles from Evonik. Varying concentrations of substances (up to 10 mg/mL citric acid) may be mixed before or after compartment printing. It was found that due to the composition of the bath, oxygen is able to diffuse through and into the compartments, allowing bacteria inside the compartments to proliferate and survive for at least one day. The results show that the viability of the bacterial cells is not affected by the yield-stress fluid, only by the citric acid. This was confirmed by printing compartments of bacteria engineered with green fluorescent protein and observing them with a confocal microscope.

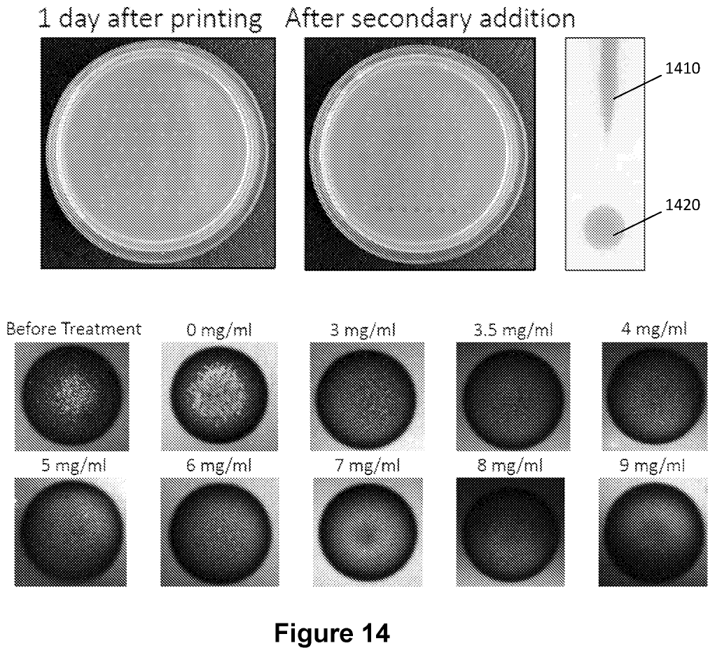

Alternatively, FIG. 14 shows a bioassay with the citric acid (drug) subsequently added via a nozzle 1410 to the compartments 1420 (P. aeruginosa in growth media). The secondary addition was made after allowing the bacteria to incubate in the compartment for one day. The results show that the bacterial cells respond to the presence of citric acid and not to the yield-stress fluid. This allows for drug efficacy to be evaluated at different points in the metabolic cycle of cells or organisms.

Similar to chemical applications, biological experiments are often extremely limited in terms of sample quantities and availability of expensive reagents. With embedded droplet printing, droplets containing biological samples are able to incubate for long periods of time but are continually directly addressable and recoverable; the droplets are stabilized without the use of molecular surfactants and are protected from exterior contaminants by the bath material. The lack of added surfactant molecules eliminates micelle formation as a mechanism for cross-droplet material transfer, an issue which has been reported to occur with droplet-based microfluidics systems. Additionally, compared to traditional droplet microfluidics assays containing microorganisms, the long term stability of the droplets of the present invention is not affected by mechanical agitation, wetting of the droplets with sidewalls, or the synthesis of new biomolecules by the microorganisms.

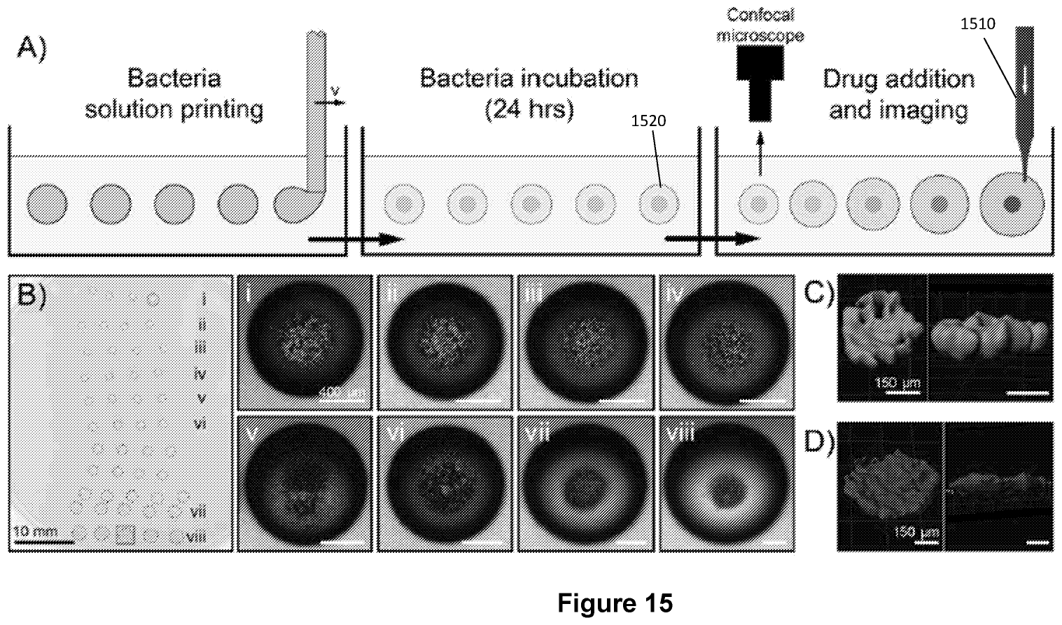

FIG. 15 shows another bioassay embodiment. Solutions of bacteria and growth media may be injected and allowed to incubate. Droplets containing aqueous growth media and the model green fluorescent protein-expressing bacteria, Pseudomonas aeruginosa, were cultured. This type of bacteria is an opportunist pathogen responsible for infections in patients under a variety of conditions. These droplets are printed inside the same oil-based yield-stress fluid described herein but with no cross-linking agent added. The droplets are left to incubate at room temperature for 24 h, during which time the bacteria consolidate at the center of the droplet. After a prescribed amount of development time, a precise volume of a drug may be injected via nozzle 1510 into each droplet 1520 of interest which may then be characterized. Here, droplets of P. aeruginosa and growth media are printed and which shows that the bacterial cells are able to survive for over 24 hours when embedded within a bath composed of fumed silica, silicone oil, and PDMS due to the permeability of the bath to oxygen. 24 hours after printing, varying volumes of a 10 mg/ml citric acid solution, a weak acid drug, and a dead stain into each of the arrayed droplets are injected. After 2 additional hours of incubation, the droplets were characterised using confocal microscopy. FIG. 15(B) shows the results as a top-down view of suspended droplets after injection of citric acid (dotted lines overlaid on droplet interfaces for clarity). Living bacteria give off a green fluorescent signal (FIG. 15(Bi-iv)) while dead bacteria give of a red fluorescent signal (FIG. 15(Bv-viii)). The roman numerals labelling each row correspond to the confocal images of a representative droplet from that row. The drug concentrations depicted (in mg/ml) are i) 0, ii) 0.5, iii) 1, iv) 2, v) 3, vi) 4, vii) 8, viii) 9. All scalebars shown in i-viii) are 400 pm. Isometric and side views of Z-stack surface reconstructions of the bacteria colony for the green circled droplet are shown in FIG. 15(D), and for the red square-outlined droplet in FIG. 15(E).

At low citric acid concentrations, nearly the entire bacterial population is alive. The bath material is permeable to oxygen and thus the bacteria are able to survive as long as there are sufficient nutrients and low concentrations of waste within the growth media droplets. At a critical concentration above 2 mg/mL of citric acid, the bacteria rapidly die. As with many conventional microfluidic techniques, in general, embedded droplet printing would allow for faster drug screening compared to traditional batch methods. If desired, the droplet contents could be transferred onto an agar plate to detect the presence of any colony-forming units.

The present invention also allows for one to easily extract fluid and samples from a given droplet for further culturing outside the bath if desired or for proteomic analysis. By combining extraction with secondary injection, one could remove waste and supply new growth media for extended periods of culturing inside the bath. In embedded droplet printing, as long as the buoyant stress does not exceed the yield stress (Equation 1), the droplets act as chambers that would be bound in size only by the overall dimensions of the yield-stress fluid bath; this means that biological samples could potentially grow inside a droplet that continually scales up in size to accommodate them via the injection of additional fluid. A unique attribute of this is that it is possible to reconfigure and merge two droplets at some selected time point by pushing them into contact using a nonwetting nozzle and then bridging them, which would not be possible with well-plate experiments. This feature could be exploited to simulate an infection in which one host grows for some time before encountering another species or multispecies studies to better understand the microbiome.

FIG. 16 provides a schematic illustration summarizing some techniques that can be used with the present invention. This allows for complex experimental scheduling to be performed and includes A) point-of-injection mixing of components via a concentric nozzle, B) injection of additional fluid after an arbitrary time period after initial printing, and C) extraction of fluid from incubated droplets. Post-printing, some bath materials may be D) permanently crosslinked to facilitate handling and characterization, while others may allow for E) removal of the yield-stress property from the bath material to enable droplet/particle recovery.

FIG. 16(A) and (B) depict two methods of mixing miscible components inside droplets—either injecting multiple components simultaneously via concentric nozzles or injecting a secondary component after the primary droplet printing process. After printed droplets have incubated for a desired amount of time, the contents can be extracted from a selected droplet as depicted in FIG. 16(C) and continue to process or characterize them outside the bath environment if desired. For the aforementioned operations, appropriate wettability of the nozzles is varied accordingly. For example, for the primary droplet printing as depicted in FIG. 16(A), consistent results are obtained when the outermost nozzle surface is preferentially wet by the bath phase. For secondary injection or extraction (FIG. 16(B) and (C), respectively), the nozzles can preferentially be wetted by the droplet phase; otherwise droplets are simply pushed around and not pierced.

FIG. 16(D) depicts method of post processing the droplets by a triggered solidification of the bath after printing. This can be achieved with a bath whose composition includes a significant proportion of polydimethylsiloxane (PDMS) as well as a crosslinking agent. After droplet printing, this oil/PDMS-based bath permanently gels with gentle heating, trapping droplets inside. Droplets are fixed in place for extended periods of time. Conceptually opposite to this, FIG. 16(E) depicts a triggered collapse of the bath microstructure (i.e., the bath loses its yield-stress property). This can be achieved with the Carbopol yield-stress fluid; the sizes of the polyacrylic acid microgels that compose this material are sensitive to pH as well as electrolyte concentration. Droplets are generated while the bath has a neutral pH (swollen microgels that are jammed). After printing, the electrolyte concentration is raised via the addition of sodium chloride, deswelling the microgels and allowing processed droplets to be recovered easily and en masse.

FIG. 17 is a schematic of an apparatus 1700 for forming compartments according to certain embodiments. The apparatus 1700 includes a nozzle 1760 and a yield-stress fluid. The yield-stress fluid can be in a bath 1710. The nozzle 1760 includes an outlet which is in contact with the yield-stress fluid. The outlet is for introducing one or more volumes into the yield-stress fluid 1710. Controller 1720 is in communication with the nozzle 1760, and possibly also with one or more components that are coupled to the nozzle 1760, such as a first translation mechanism that is configured to displace the nozzle 1760 relative to the yield-stress fluid 1710. Controller 1720 may also be in communication with a second translation mechanism that is coupled to the yield-stress bath 1710 and that is configured to displace the yield-stress bath 1710 relative to the nozzle 1760, such as a translation stage 1750. The displacement allows for the introduction of one or more volumes (from the outlet of the nozzle 1760) into the yield-stress fluid 1710 to form the one or more compartments. In this regard, the nozzle can be moved while the yield-stress fluid is stationary, or the yield-stress fluid can be moved while the nozzle is stationary, or both the nozzle and yield-stress fluid can be moved.

The one or more volumes can be provided by a microfluidic system 1740. The microfluidic system can be provided in communication with an inlet of the nozzle. The microfluidic system can be modulated to provide the one or more volumes. Alternatively, the flow of the input fluid can be selectively perturbed to form one or more volumes via a pump 1730.

The yield-stress bath 1710 can optionally be located on a translation stage 1750. The translation stage 1750 allows the displacement of the yield-stress fluid relative to the nozzle.

Accordingly, there is also provided an apparatus for forming one or more compartments, including: a) a nozzle including an outlet, the outlet for introducing one or more volumes; b) a yield-stress fluid, the yield-stress fluid in contact with the outlet of the nozzle; and c) a controller configured to displace the nozzle and/or the yield-stress fluid relative to each other to introduce one or more volumes into the yield-stress fluid to thereby form one or more compartments from the one or more volumes.

The apparatus can further include a controller configured to selectively perturb a flow of an input fluid for forming one or more volumes.

In certain embodiments, the selective perturbation of the flow of the input fluid is synchronised to the displacement the nozzle and/or the yield-stress fluid relative to each other. In certain embodiments, the selective perturbation of the flow of the input fluid is a periodic variation of the flow of the input fluid. In other embodiments, the controller configured to selectively perturb the flow is a compartment/volume generator. The compartment generator can rely on Rayleigh instability, or can be based on a stop-flow modulator.

The apparatus may further include a microfluidic system in communication with an inlet of the nozzle. Alternatively, the apparatus may further include a pumping system in communication with the inlet of the nozzle. The same or a separate microfluidic system and/or pumping system can also be in communication with the yield-stress fluid.

In certain embodiments, the yield-stress fluid is contained in a vessel.

In certain embodiments, nozzle is displaceable relative to the vessel. Alternatively, the vessel is displaceable relative to the nozzle. In certain embodiments, the displacement is in Cartesian coordinates. In some embodiments, the yield-stress fluid is mounted on an X, Y-axis motion-controlled stage and one or more nozzles mounted on a Z-axis motion controller. The motion controllers can be programmed or operated to move in a determined pattern at a pre-determined and controlled movement rate.

High degrees of particle uniformity and sphericity are key manufacturing targets for enhancing powder flowability and processability. As an example for further improving suitability for manufacturing applications, a platform that enables the continuous production of embedded droplets and for use to produce flowable pharmaceutical particles is exemplified. The setup in FIG. 18 is for the “direct-write” embodiment. Shown in FIG. 18A, continuous production can be achieved via a (walled) conveyor belt 1810 which carries a film of yield-stress fluid 1820 into which a robotic arm 1830 injects an immiscible droplet phase. Here the yield-stress fluid 1820 is deposited and spreads on the belt which carries a thin film of the fluid relatively slowly away from the deposition point. The yield-stress fluid 1820 is deposited upstream from a reservoir 1840 (supplied to the conveyor belt 1810 via a peristaltic pump 1850), enabling an unlimited printing space for droplets that are collected downstream. The input fluid can be provided by a syringe pump 1860. The robotic arm 1830 holding an inlet nozzle translates at a much faster speed (>10×) in the transverse direction, generating droplets. The formed embedded droplets can be collected in a sieve and waste container 1870. This platform expands the functionality of embedded droplet printing towards continuous materials manufacturing. With this platform, the production throughput of embedded pharmaceutical particles can be increased and thus rapidly obtain enough sample to characterize the rheology of these powders. This example shows that the present invention can be applied to the antisolvent crystallization of pharmaceuticals.

In particular, the above example was setup using a robot arm and conveyor belt from Dobot and attaching a glass capillary nozzle with a nominal tip diameter of 30 μm to the end of the arm. Elastic bands are placed around the conveyor belt to create side walls of a nominal height of 6 mm and a center channel with a nominal width of 50 mm. The length of the belt is approximately 700 mm. Nozzles are connected to glass syringes mounted in a Harvard Apparatus syringe pump. The yield-stress fluid is Carbopol 980 in water, prepared to a concentration of 0.08 wt %. This fluid is deposited on the conveyor belt via a BT100S peristaltic pump through a flexible 1 cm silicone tube. Antisolvent crystallization using a solution of 50 mg/mL Naproxen (a model hydrophobic active pharmaceutical ingredient), 20 mg/mL ethyl cellulose (a model excipient) in ethyl acetate was performed.

FIG. 18B depicts the continuous printing setup schematically. When performing antisolvent crystallization, it was found that sufficient time must be allowed for the solvent (ethyl acetate) to diffuse into the antisolvent yield-stress fluid, leaving behind a crystallized particle. Thus, without wanting to be bound by theory, the characteristic diffusion time (diameter squared divided by the diffusivity, D) for a droplet with radius, r, should be less than the time it takes for the conveyor belt to travel its length, LB, at a speed of VB. A printing path was programmed that generates parallel lines of droplets with a constant spacing, δ, which is equal to the distance the belt travels during the time it takes for the nozzle to move across the belt width, WB, traveling at a speed, VP.

To prevent the droplets from interacting, it requires δ to be greater than or equal to two times the radius of a droplet. Combined, these two conditions set the following boundaries on the belt speed in terms of the printing parameters, droplet size, and geometry of the belt setup:

$$\frac{2 {rV}_{P}}{{W}_{B}} \leq {V}_{B} \leq \frac{{DL}_{B}}{{( 2 r )}^{2}} \qquad ( 3 )$$

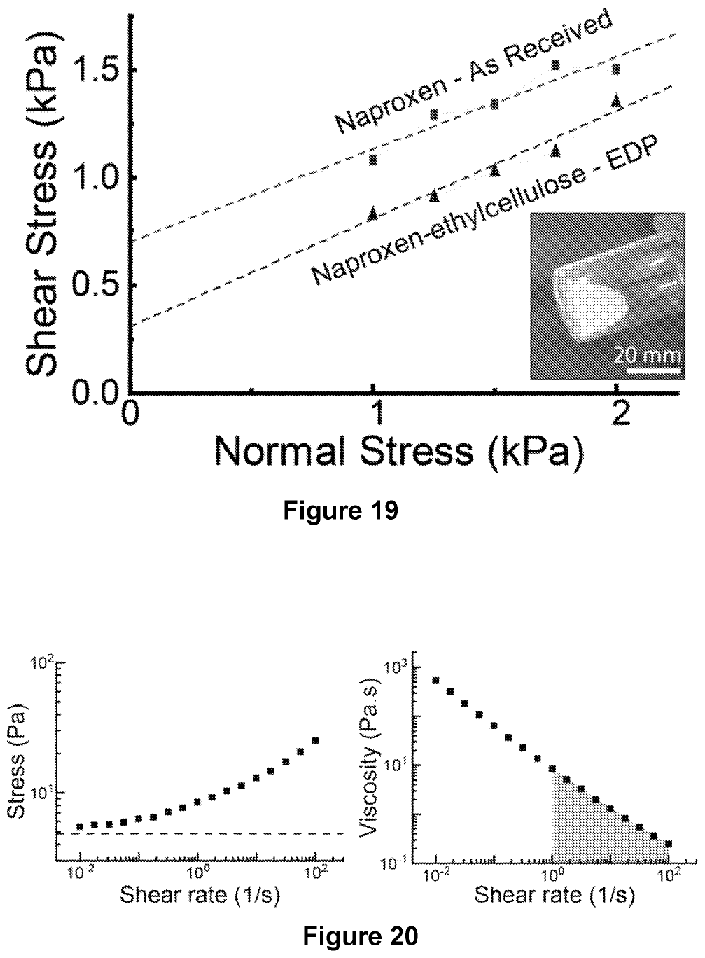

The robot arm 1830 was programmed to print at a speed of 2000 mm/min and the syringe pump to inject at a volumetric flowrate of 200 μL/min, producing droplets approximately 800 microns in diameter at a total mass throughput of approximately 840 mg/h. For ethyl acetate diffusing in water, this results in a belt speed range of 0.8 to 1 mm/s to satisfy Equation 3. Choosing 1 mm/s, we produce the several mL of powder needed for rheological characterization in approximately 1 hour, significantly less time than would have been required with the semi-batch method. Particles were recovered from the yield-stress fluid bath via salt addition, and dried overnight before characterizing. The pharmaceutical powder is shown in the inset of FIG. 19. The incipient shear stresses for flow as a function of applied normal stress are compared to as-received Naproxen powder in FIG. 19. Powder rheometry experiments was performed on an FT4 rheometer from Freeman Technology using a 1 mL shear cell fixture and a pre-consolidation normal stress of 3 kPa. On average, the incipient shear stress of the embedded droplet powders is reduced to approximately 75% of the as-received powder. A linear fit to the data results allows one to obtain the cohesion parameter from the y-intercept; a reduction of over 50% was observed, from 0.68 kPa to 0.31 kPa. Thus, significant improvement in rheology measurements of particles produced via embedded droplet printing can be obtained.

The platform can help pave the way for applying the unique functionality of embedded droplet printing to many continuous processes, including particle manufacturing. For pharmaceuticals, the use of similar platforms to enable precise, rapid, customized, and distributed drug manufacturing is envisioned.

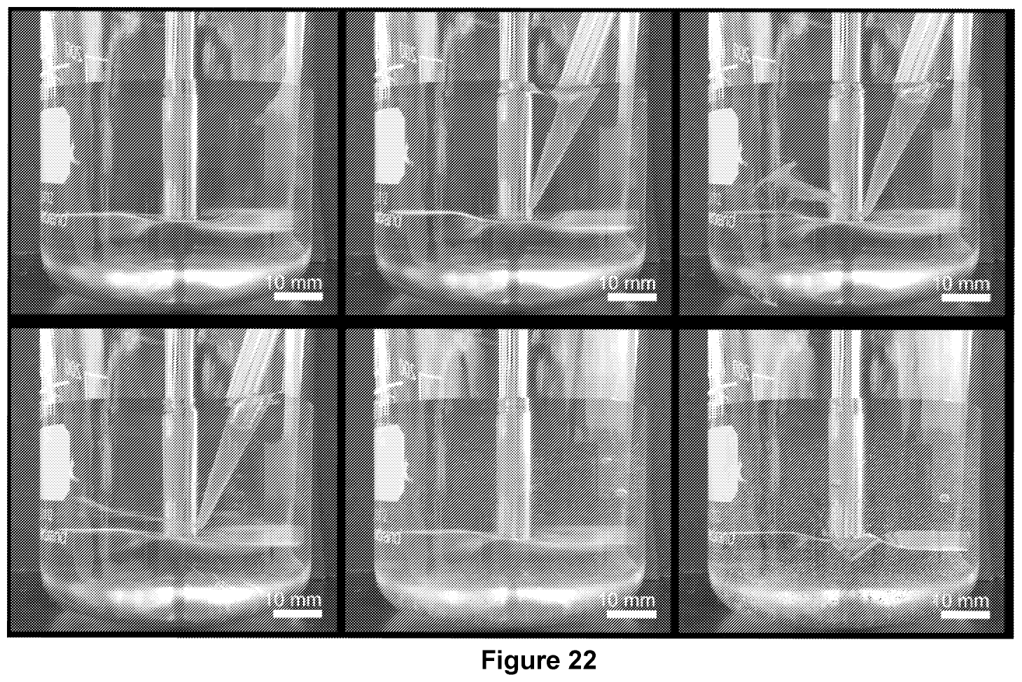

Images of various phases of performance of another example method according to the present invention are shown in FIG. 22. As illustrated in the images (from left to right, top to bottom), the generation of compartments of drug solution via mechanical shearing of a stream submerged in a yield-stress fluid can be achieved. The impeller is submerged in yield-stress fluid and rotated at a high speed. An injected stream of a partially miscible drug solution is broken apart into droplets by the motion induced by the impeller. When the rotation is stopped, the droplets are embedded in place and solidify. Additionally, the yield-stress fluid can be in communication with a reservoir and extraction tube to continuously remove bath material along with embedded droplets while adding fresh bath material. Similarly, the drug solution could be added continuously or periodically. This method has the advantage of a high processing throughput and maintains the quiescent yield-stress fluid environment of the typical embedded droplet printing.

### EXAMPLES

### Materials and Methods

### Yield-Stress Fluid Bath Preparation.

Materials. Carbopol 980 (cross-linked polyacrylic acid particles) was obtained from Lubrizol. Ultrapure water (18.2 MΩ) was obtained from a Sartorius H2OPRO-DI-T Arium

Pro purifier. Sodium hydroxide (221465) and silicone oil (317667) were purchased from Sigma-Aldrich. Hydrophilic silica nanoparticles (AEROSIL 200) were obtained from Evonik Industries. PDMS and curing agent (Sylgard 184) were obtained from Dow Corning.

Water-based yield-stress fluid. Aqueous jammed suspensions of polymer microgels were prepared by mixing Carbopol 980 powder in ultrapure water at a concentration of 0.1 wt %. This solution was mixed for 30 min before being neutralized to pH 7 using a 1 M sodium hydroxide solution. This solution was placed in a vacuum chamber to remove air bubbles.

Oil-based yield-stress fluid. Silica nanoparticle oleogels were prepared by adding PDMS and then silicone oil to hydrophilic silica powder to achieve a final composition of 4 wt % silica, 48 wt % PDMS, 48 wt % silicone oil. This solution was first mixed vigorously by hand and then placed on an IKA Eurostar 40 digital stand mixer with a four-bladed propeller stirrer (R1342) to mix for 15 min at 2,000 rpm. If the bath was to be cross-linked, immediately prior to droplet printing, the yield-stress fluid and curing agent were mixed in a 10:1 ratio (wt/wt) and placed in a vacuum chamber to remove air bubbles. For consistent shear history and printing conditions, even if no cross-linker was to be added, immediately prior to droplet printing, this bath material would be vigorously mixed and the bubbles removed.

Motorized Platform System. For computer control of our droplet generation, we use the motorized platform of a 3Drag open-source 3D printer purchased from Open Electronics with an Arduino running Marlin firmware that accepts G-code commands through the freely available software Repetier. To the stationary frame of this platform, we mount nozzles that are either flattipped needles or surface-treated glass capillaries. Nozzle surfaces were modified to be preferentially wet by either the bath or the droplet phase, depending on the situation as discussed in Toolbox of Embedded Droplet Printing. For hydrophobic modification, nozzles were plasma cleaned and then treated with the commercial product Rain-X according to the manufacturer instructions. For hydrophilic modification, nozzles were plasma cleaned and submerged in ultrapure water for storage before printing. For secondary injection and extraction, prepulled glass micropipettes with a nominal tip diameter of 20 μm from FIVEphoton Biochemicals were used. Nozzles are connected via poly(tetrafluoroethylene) tubes (inner diameter 1 mm) purchased from Cole Parmer to 2.5-mL glass syringes (Hamilton GASTIGHT) purchased from Sigma-Aldrich mounted in Harvard Apparatus syringe pumps (PHD ULTRA 70-3007). We use custom-made 3D printed mounts to hold containers of yield-stress fluid in place on the motorized stage.

### Characterization of Model System.

Materials. Light mineral oil (330779) was purchased from Sigma-Aldrich. For FIGS. 2 and 5, a commercial red food dye was added to the mineral oil for clarity. In all other cases, the mineral oil was used as received. Flat-tipped stainless steel needles were purchased from Taobao in nominal sizes of (inner diameter:outer diameter in millimeters) 0.21:0.42, 0.41:0.72, 0.44:0.8, 0.72:1.08, and 1.5:1.8 mm. Glass capillary tubes were purchased from VWR in nominal sizes of 0.67:1 and 1:1.5 mm and were plasma cleaned immediately prior to droplet printing. Stainless steel needles were not plasma cleaned. Sixty-millimeter diameter Petri dishes were purchased from Thermo Scientific. Carbopol solution (0.1 wt %) was prepared as described above.

Single-phase droplet printing and characterization. Nozzles were mounted to the stationary frame of the motorized platform described above and connected to a gas-tight glass syringe filled with mineral oil that was placed in a Harvard syringe pump. Petri dishes were filled with Carbopol solution to a thickness of ˜1 cm and mounted on the motorized stage. The nozzle was positioned at a nominal height of 5 mm above the bottom of the dish. The platform was programmed to move in a serpentine pattern at progressively faster speeds starting at 100 mm/min and ending at 3,000 mm/min, with 2.5 mm between rows. Row spacing of 2.5 mm and layer spacing of 5 mm were used to calculate the volumetric density of droplets. Just after nozzle translation initiated, the mineral oil was injected at a volumetric flow rate of 50 or 100 μL/min via the syringe pump. Droplets were imaged on an inverted Olympus IX71 microscope with a 4x or 10x objective. Droplet size and linear density measurements were made using ImageJ .

Rheological characterization of bath material. Rheological characterization of steady flow properties was performed on an AR-G2 combined motor/transducer rotational rheometer from TA Instruments, using parallel-plate geometry with a diameter of 40 mm and a Peltier temperature controller set to 25° C. A range of shear rates was applied from high to low and the apparent steady stress was recorded and fitted to a Herschel-Bulkley model using Origin 2019 and used to calculate the corresponding viscosity as a function of the shear rate.

Spherical Crystallization of Pharmaceutical Particles. Materials. Naproxen (N8280), ethyl cellulose (viscosity 10 cP 200689), and sodium chloride (S5886) were purchased from Sigma-Aldrich. Ethyl acetate (HiPerSolv

CHROMANOFORM for HPLC, ≥99.8%) was purchased from VWR International, LLC, and used as received. Ultrapure water (18.2 MΩ at 25° C.) was obtained from a Sartorius H2OPRO-DI-T Arium Pro purifier. Cell strainers with a mesh size of 100 μm were purchased from Fisher Scientific. Carbopol solution (0.1 wt %) was prepared as detailed above.

Production of crystallized particles. The drug-loaded injected phase was prepared by dissolving 50 mg of naproxen and 20 mg of ethyl cellulose per milliliter of ethyl acetate. This solution was delivered at a volumetric flow rate of 50 μL/min through a flat-tipped needle of nominal inner diameter 0.44 mm and nominal outer diameter 0.8 mm, submerged in the Carbopol bath material contained by a rectangular plastic box. The motorized platform was programmed to move at a constant linear speed of 1,000 mm/min in a multilevel serpentine pattern. After suspended droplets were generated, the bath and droplets were left undisturbed for at least 20 min (double the characteristic diffusion time estimated in the main text) for crystallization to proceed. To collapse the

Carbopol material, a volume of 1 M NaCI solution approximately equal to 5% of the volume of the bath was added followed by agitation of the bath. Particles were collected on a cell strainer, rinsed three times with ultrapure water, and vacuum dried at room temperature for at least 12 h prior to structural and polymorphic characterization.

Characterization of pharmaceutical particles. All samples were prepared for scanning electron microscopy on conventional stubs with a silicon wafer surface and were coated with ˜10 nm of platinum by sputter coating. A field-emission scanning electron microscope (JEOL JSM-6700F) at 5 kV accelerating voltage was used to image the particles. Polymorphic characterization of particles was analyzed using differential scanning calorimetry (DSC) and powder X-ray diffraction (PXRD) to examine their crystallinity. An X-ray diffractometer (Bruker; D8 Advance) was operated at 40 kV, 30 mA, and at a scanning rate of 1.06°/min over a range of 2θfrom 2.5-30° , using a Cu radiation wavelength of 1.54 Å. For DSC, a Mettler Toledo DSC 882 apparatus was used. Approximately 5 mg of sample was crimped in a sealed aluminum pan and heated at 5 ° C/min in the range of −20 to 180 ° C. using an empty sealed pan as a reference.

Embedded Chemical Reaction Chambers. Materials. Silver nitrate (99.9%) from Strem Chemicals and hydrazine hydrate (50 to 60%), sodium borohydride (98%), poly(vinyl alcohol) (molecular weight 67,000), and sodium citrate tribasic dihydrate (99%) from Sigma-Aldrich Co. Ltd. were used as received without any further purification. Ultrapure water (18.2 MQ at 25 ° C.) was obtained from a Milli-Q purifier. Petri dishes were purchased from Thermo Scientific.

Synthesis of silver seed solution. A total of 2 mL of 1 mM silver nitrate solution and 2 mL of 1 wt % aqueous poly(vinyl alcohol) solution are pipetted into a 20-mL glass bottle with a magnetic stirrer. The solution is stirred at 1,400 rpm tomix the precursor solution. After that, 2 mL of 2.5mMsodium borohydride solution is added rapidly into the glass bottle. The product is left to stir for 15 min.

Synthesis of silver nanoparticles. Aqueous growth solution consists of 2 mL of seed solution, 3 mL 100 mM sodium citrate solution, 5 mL of 1 wt % poly(vinyl alcohol) solution, and 5 mL of 40 mM hydrazine solution. A total of 10 mL of 1 mM silver nitrate solution is prepared as the precursor solution. These two solutions are filled into separate gas-tight Hamilton syringes. The solutions are delivered by using two syringe pumps (Harvard PHD ULTRA) into a coaxial glass capillary microfluidic device with the outermost surface (1.5 mm outer diameter) treated with Rain-X according to the manufacturer instructions to be hydrophobic and more readily wet by the bath material. The growth solution flows through the inner channel while the fluid in the outer channel is the precursor solution. Near the tip of the device, the two solutions meet and are printed as a single droplet in the oil-based yield-stress bath that contains cross-linking agent as described above. Before printing, the oil-based yield-stress bath was conditioned as described above in a Petri dish at a thickness of ˜1 cm. To produce silver nanoparticles of various sizes in each droplet, a ramp program is used, where the growth solution flow rate is decreased from 100 to 55 μL/min and the precursor solution flow rate is increased from 5 to 50 μL/min.

Characterization of silver nanoparticles. To prepare for TEM imaging, the silver nanoparticles solution in the printed droplet is extracted from the bath via a pipette and diluted with ultrapure water. A drop of this diluted sample is placed onto a 200-mesh copper grid, which is dried overnight and analyzed using TEM (JOEL 2010; accelerating voltage 200 K). In addition, the optical properties of these silver nanoparticles are identified via Resonon Hyperspectral Imaging Systems (Pika L with Backlight stage). Before characterization, the Petri dish containing the bath and droplets was placed on a hot plate at 35° C. for several hours to cross-link the bath. Droplets were characterized within 24 h to avoid any observed evaporation through the now-porous PDMS material. With this hyperspectral system, the intensity of the light passing through each silver nanoparticle-containing droplet is recorded and used to compute the absorbance of the silver nanoparticle solution. Droplets of the same size containing only ultrapure water were used to obtain a reference absorbance.

### Embedded Biological Assays.

Biofilm formation in microarrays. P. aeruginosa mucA strains, which were fluorescently tagged with green fluorescent protein (eGFP), were used to make the microprinted droplet arrays of biofilms. Overnight cultures of P. aeruginosa strains were grown in

Luria—Bertani broth (5 g/L NaCI, 5 g/L yeast extract, 10 g/L tryptone) at 37° C. under shaking conditions (200 rpm). The overnight P. aeruginosa culture was diluted to an optical density at 600 nm (OD600) of 0.4, and 1 mL of the culture was taken in a glass Hamilton syringe and printed into the oil-based yield-stress bath at a volumetric flow rate of 50 μL/min via a syringe pump (Harvard PHD ULTRA). The motorized platform was programmed to move at a constant linear speed of 500 mm/min in a serpentine pattern. The arrayed microdroplets of bacteria culture were then allowed to grow for 24 h to form blobs of biofilm. Treatment, staining, and imaging. Citric acid (Sigma-Aldrich) at a concentration of 10mg/mL prepared using LB medium was added to each droplet in varying volumes via a hydrophilically modified glass capillary connected to a syringe pump so that the final concentration in each droplet ranging from 0 to 9 mg/mL propidium iodide (PI) (Sigma-Aldrich) was added to citric acid to a final concentration of 60 μM to visualize the dead bacteria in biofilms. The citric acid—PI mixture was added to the microarrays and incubated for 2 h to allow for the action of citric acid on the bacteria and staining of the dead cells by PI. The droplets were then imaged using a

FluoView 1000 confocal microscope (Olympus) with a 4x objective. Three image channels, GFP 488, Alexa 594, and bright field, were acquired from each droplet. The 3D image stacks of the representative droplets were also acquired to visualize the biofilm structure within the droplets before and after treatment with the citric acid.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method for forming one or more compartments in a yield-stress fluid, including: a) introducing one or more volumes from an outlet of a nozzle into a yield-stress fluid, the outlet of the nozzle being in contact with the yield-stress fluid; and b) displacing the nozzle and/or the yield-stress fluid relative to each other to thereby form one or more compartments in the yield-stress fluid, the one or more compartments being formed by, or formable from, the one or more volumes.

2. The method according to claim 1, further including a step of selectively perturbing a flow of an input fluid for forming one or more volumes.

3. The method according to claim 2, wherein the selective perturbation of the flow of the input fluid is synchronised to the displacement of the nozzle and/or the yield-stress fluid relative to each other.

4. The method according to claim 2 or 3, wherein the selective perturbation of the flow of the input fluid is a periodic variation of the flow of the input fluid.

5. The method according to any of claims 1 to 4, wherein the one or more compartments is, or are, one or more droplets, and the one or more droplets are suspended in the yield-stress fluid.

6. The method according to any of claims 1 to 5, further including a microfluidic system in communication with an inlet of the nozzle for supplying the input fluid.

7. The method according to any of claims 1 to 5, further including a flow system in communication with the yield-stress fluid for supplying the yield-stress fluid as a continuous flow to the outlet of the nozzle.

8. The method according to any of claims 1 to 7, wherein the one or more compartments is, or are, non-miscible with the yield-stress fluid.

9. The method according to any of claims 1 to 8, wherein the yield-stress fluid is at rest.

10. The method according to any of claims 1 to 9, wherein the yield-stress fluid is contained in a vessel. 5

11. The method according to any of claims 1 to 10, wherein the vessel is displaceable relative to the nozzle.

12. The method according to claim 11, wherein the displacement is in Cartesian coordinates.

13. The method according to any of claims 1 to 12, wherein the yield-stress fluid has a yield-stress value of about 0.1 Pa to about 10 Pa.

14. The method according to any of claims 1 to 13, wherein the yield-stress fluid has a surface tension of about 5 mN/m to about 75 mN/m.

15. The method according to any of claims 1 to 14, wherein the yield-stress fluid has critical shear rate \dot{y}_{c} of about 0.01 1/s to 1000 1/s, and a flow index n of about 0.25 to about 1.

16. The method according to any of claims 1 to 15, wherein the yield-stress fluid has a characteristic thixotropic timescale of about 0 seconds to about 10 seconds.

17. The method according to any of claims 1 to 16, wherein yield-stress fluid is selected from polydimethylsiloxane, silicone oil, colloidal particles in water or oil, diblock or triblock copolymers in water or oil, microcellulose, xanthum gum, 0.1wt % Carbopol and a combination thereof.

18. The method according to any of claims 1 to 17, wherein the one or more compartments is, or are, suspended within the yield-stress fluid at least 1 mm below a surface of the yield-stress fluid.

19. The method according to any of claims 2 to 18, wherein the input fluid is a liquid.

20. The method according to any of claims 1 to 19, wherein the one or more volumes in the one or more compartments is, or are, diffusible out from the one or more compartments for use in crystallisation.

21. The method according to any of claims 1 to 20, wherein each of the one or more compartments has a different composition for following a progress of a chemical reaction.

22. The method according to any of claims 1 to 21, wherein each of the one or more compartments includes a microorganism for use in bioassays.

23. An apparatus for forming one or more compartments, including: a) a nozzle including an outlet, the outlet for introducing one or more volumes; b) a yield-stress fluid, the yield-stress fluid in contact with the outlet of the nozzle; and c) a controller configured to displace the nozzle and/or the yield-stress fluid relative to each other to introduce one or more volumes into the yield-stress fluid to thereby form one or more compartments from the one or more volumes.

24. The apparatus according to claim 23, further including a controller configured to selectively perturb a flow of an input fluid for forming one or more volumes.

25. The apparatus according to claim 24, wherein the selective perturbation of the flow of the input fluid is synchronised to the displacement the nozzle and/or the yield-stress fluid relative to each other.

26. The apparatus according to claim 24 or 25, wherein the selective perturbation of the flow of the input fluid is a periodic variation of the flow of the input fluid.

27. The apparatus according to any of claims 23 to 26, further including a microfluidic system in communication with an inlet of the nozzle.

28. The apparatus according to any of claims 23 to 27, wherein the yield-stress fluid is contained in a vessel.
